(19) United States
(12) Patent Application Publication
(10) Pub. No.: US 2003/0225036 A1
(43) Pub. Date: Dec. 4, 2003

(54) Non-amidine containing protease inhibitors

(76) Inventors: Aleksandr Kolesnikov, San Francisco, CA (US); Roopa Rai, San Carlos, CA (US)

Correspondence Address: Celera, 180 Kimball Way, South San Francisco, CA, 94080, US

(21) Appl. No.: 10/221,567

(22) PCT Filed: Mar. 20, 2001
(86) PCT No.: PCT/US01/08839

(30) Foreign Application Priority Data
Mar. 20, 2000 (US) 60190731

(51) Int. Cl. A61K 31/675; C07D 473/00; C07D 471/02; A61K 31/52; A61K 31/519

(52) U.S. Cl. 514/81; 514/252.16; 514/253.04; 514/253.03; 514/248; 514/249; 514/263.2; 514/265.1; 514/300; 514/303; 514/292; 544/235; 544/244; 544/277; 544/362; 546/23; 546/85; 546/113; 546/118

(57) ABSTRACT

The present invention provides novel compounds of the Formula (I) its prodrug forms, or pharmaceutically acceptable salts thereof. Preferred (I) compounds of the present invention comprise a pyrrolo pyridinyl, pyrrolo pyrimidinyl or indole nucleus. The compounds of this invention are inhibitors of Factor Xa (FXa), Factor VIIa (FVIIa) and/or serine proteases, Urokinase (uPA), and have utility as anti-coagulants for the treatment or prevention of thromboembolic disorders in mammals and as anticancer agents.

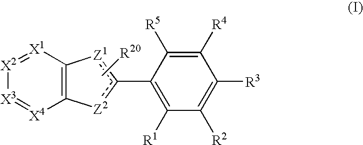

### FIELD OF INVENTION

The present invention relates to non-amidine containing novel protease inhibitors.

### BACKGROUND OF THE INVENTION

Factor Xa (herein after “FXa”), the converting enzyme of pro-thrombin to thrombin, has emerged as an alternative (to thrombin) target for drug discovery for thromboembolic disorders. A variety of compounds have been developed as potential FXa inhibitors.

Kunitada and Nagahara in Current Pharmaceutical Design, 1996, Vol. 2, No.5, report amidinobenzyl compounds as FXa and thrombin inhibitors. Disclosed in U.S. Pat. No. 5,576,343 are aromatic amidine derivatives and salts thereof, as reversible inhibitors of FXa. These compounds comprise amidino substituted indolyl, benzofuranyl, benzothienyl, benzimidazolyl, benzoxazoyl, benzothiazolyl, naphthyl, tetrahydronaphthyl and indanyl groups, attached to a substituted phenyl ring by an alkylene group having from 1 to 4 carbon atoms.

Inhibitors of blood-clotting enzymes such as Factor Xa and Factor VIIa, are also known to be inhibitors of serine proteases such as Urokinase (uPA). Urokinase-type plasminogen activator (uPA) is one class of protease that plays a significant role in the progression of cancer. Inhibitors of uPA have been postulated to be of therapeutic value in treating cancer.

In spite of the above discussed efforts, desirable treatment of cancer and thromboembolic disorders still remains elusive. There is thus a need for new compounds that will be effective in inhibiting blood-clotting enzymes such as FXa and serine proteases, such as Urokinase. Keeping these needs in mind, the present invention provides novel inhibitors as discussed below.

### SUMMARY OF THE INVENTION

Keeping the above discussed needs in mind, the present invention provides compounds of Formula I:

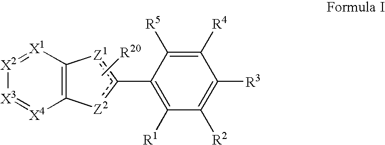

its prodrug forms, or pharmaceutically acceptable salts thereof, wherein

R¹ represents OH, halogen, COOH, COO—C₁₋₄ alkyl, O—(CH₂)₀₋₁—Ar, N(R¹⁰)₂, CH₂OR¹⁰,C₁₋₆ halogenated alkyl, O—(CH₂)₁₋₄—CO—N(R¹⁰)₂, SC₁₋₄ alkyl, NHSO₂C₁₋₄alkyl, SO₂—OH, O—SO₂—OH, O—C₁₋₄ alkyl, O—C₃₋₉ cyclo alkyl, O—SO₂—O—C₁₋₄ alkyl, OP(O) (OH)₂, or OPO₃C₁₋₄ alkyl; R², R³, R⁴, and R⁵ independently at each occurance represent H, SH, OR¹⁰, halogen, COOR¹⁰, (CH₂)₀₋₆—CONR¹¹R¹², optionally substituted aryl, optionally substituted heterocyclyl, C₄₋₁₄ cycloalkyl-C₁₋₄ alkyl, C₁₋₄ alkyl aryl, optionally substituted C₁₋₁₄ straight chain, branched or cyclo alkyl, O— (CH₂)₂₋₆—NR¹⁰—(CH₂)₀₋₃—R²⁴, NR¹⁰R²⁴, (CH₂)₁₋₆—NR³³R³⁴, (CH₂)₁₋₆—COOR³³, O—(CH₂)₁₋₃—CO—het, O—(CH₂)₁₋₂—NH—CO-aryl, O—(CH₂)₁₋₂—NR¹⁰—CO—NR¹⁰R³³, (CH₂)₁₋₄—CONR¹⁰(CH₂)₁₋₄-heterocyclyl, O—(CH₂)₀₋₂—C(O)—NR³³R³⁴, O—(CH₂)₁₋₄—COOR¹⁰, O—(CH₂)₁₋₃-het-R³², O-optionally substituted cycloalkyl, O—(CH₂)₁₋₄-NR¹⁰—COO-t-butyl, O—(CH₂)₁₋₄—NR¹⁰R³³, O—(CH₂)₁₋₄—NR¹⁰—C(O)—C₀₋₃-alkyl-optionally substituted aryl, O-substituted cycloalkyl, O—(CH₂)₀₋₆-optionally substituted aryl, (CH₂)₁₋₄—NH—C(O)O— (CH₂)₁₋₄-PhR¹³R¹⁴, NO₂, O—(CH₂)₀₋₄—C(O)—NH-tetrahydro carboline, NR¹⁰R²⁸, O—(CH₂)₁₋₃-optionally substituted het, CH₂COOCH₃, CH═CH—COOCH₃, 5-amidino benzimidazole, SO₂—N(R¹⁰)₂,

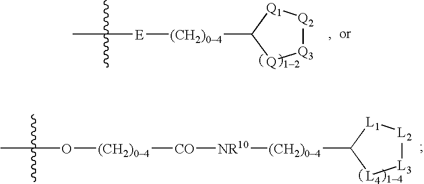

alternatively R² and R³, R³ and R⁴ or R⁴ and R⁵ can be taken together to form

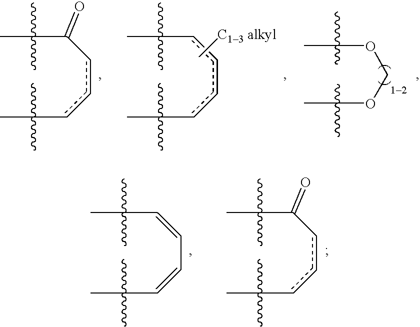

X¹ represents C—R⁶, N or N—O;

X² represents C—R⁷, N or N—O;

X³ represents C—R⁸, N or N—O;

X⁴ represents C—R⁹, N or N—O;

Z¹ and Z² independently at each occurance represent C or N;

R⁶, R⁸ and R⁹ independently at each occurance represents H, halogen, cyano, C₁₋₄ alkyl, C₁₋₄ halogenated alkyl, NO₂, O-aryl or OR¹¹, CF₃, OC₁₋₄ alkyl, (CH₂)₀₋₄-aryl, (CH₂)₀₋₄-heteroaryl, or (CH₂),₀₋₄-heterocyclyl;

R⁷ represents NH₂, NHR¹⁰, N(R¹⁰)₂, NHSO₂—C₁₋₁₄ alkyl, NHSO-aryl, OH, NHCO—C₁₋₁₄ alkyl, NHNH₂, NHOH, NHCO—C₁₋₁₄ alkyl, NR¹⁰NH₂, NHN(R¹⁰)₂, NH(C═NH)NH₂, NH(C═O)N(R¹⁰)₂; alternatively R⁶ and R⁷, R⁷ and R⁸, R⁸ and R⁹, along with the respective carbon atoms to which they are attached, can be taken together to represent a 5 to 10 atom saturated, partially saturated or aromatic, carbocyclic or heterocyclic ring structure substituted with R⁴¹;

R¹⁰ independently at each occurance represents H, (CH₂)₀₋₂-aryl, C₁₋₄ halo alkyl, or C₁₋₁₄ straight chain, branched or cyclo alkyl, and alternatively, when one atom is substituted with two R¹⁰ groups, the atom along with the R¹⁰ groups can form a five to 10 cycloalkyl, heterocyclyl or aryl group;

R¹¹ and R¹² independently at each occurance represent H or C₁₋₄ alkyl, (CH₂)₁₋₄—OH, (CH₂)₁₋₄ alkyl, (CH₂)₀₋₄-aryl, (CH₂)₁₋₄—(R¹⁰)₂;

R²⁰ represents R²⁴, C₁₋₄-alkyl, (CH₂)₁₋₃-biphenyl, (CH₂)₁₋₄-Ph-N(SO₂—C₁₋₂-alkyl)₂, (CH₂)₁₋₄—NH—C(O)—R²⁴, (CH₂)₁₋₄—NH—SO₂—R²⁴, halogen, COOR¹⁰, (CH₂)₁₋₄-Ph-N(SO₂—C₁₋₂alkyl), (CH₂)₁₋₄—NR—C(O)—R²⁴, (CH₂)₁₋₄—NR¹⁰—SO₂—R²⁴, (CH₂)₁₋₄-het, (CH₂)₁₋₄—CON(R¹⁰)₂, (CH₂)₁₋₄—N(R¹⁰)—C(O)—NR¹⁰R²⁴, (CH₂)₁₋₄—N(R¹⁰)—C(S)—NR¹⁰R²⁴ , or (CH₂)₁₋₃—COOH;

R²⁴ represents R¹⁰, (CH₂)₁₋₄-optionally substituted aryl, (CH₂)₀₋₄OR¹⁰, CO— (CH₂)₁₋₂—N(R¹⁰) ₂, CO(CH₂)₁₋₄—OR¹⁰, (CH₂)₁₋₄—COOR¹⁰, (CH₂)₀₋₄—N(R¹⁰)₂, SO₂R¹⁰, COR¹⁰, CON(R¹⁰)₂, (CH₂)₀₋₄-aryl-COOR¹⁰, (CH₂)₀₋₄-aryl-N(R¹⁰)₂, or (CH₂)₁₋₄-het-aryl;

R²⁸ represents (CH₂)₁₋₂-Ph-O—(CH₂)₀₋₂-het-R³⁰, C(O)-het, CH₂-Ph-CH₂-het-(R³⁰)₁₋₃; (CH₂)₁₋₄-cyclohexyl-R³¹, CH₂-Ph-O-Ph-(R³⁰)₁₋₂, CH₂—(CH₂OH)-het-R³⁰, CH₂-Ph-O-cycloalkyl-R³¹, CH₂-het-C(O)—CH₂-het-R³⁰, or CH₂-Ph-O— (CH₂)—O-het-R³⁰;

R³⁰ represents SO₂N(R¹⁰)₂, H, NHOH, amidino, or C(═NH)CH₃;

R³¹ represents R³⁰, amino-amidino, NH—C(═NH)CH₃ or R¹⁰;

R³² represents H, C(O)—CH₂—NH₂, or C(O)—CH(CH(CH₃)₂) —NH₂;

R³³ and R³⁴ independently at each occurance represent R¹⁰, (CH₂)₀₋₄—Ar, optionally substituted aryl, (CH₂)₀₋₄ optionally substituted heteroaryl, (CH₂)₁₋₄—CN, (CH₂)₁₋₄—N(R¹⁰)₂, (CH₂)₁₋₄—OH, (CH₂)₁₋₄—SO₂—N(R¹⁰)₂;

alternatively, R³³ and R³⁴ along with the nitrogen atom that they are attached to forms a 4 to 14 atom ring structure selected from tetrahydro-1H-carboline; 6, 7-Dialkoxyoxy-2-substituted 1,2,3,4-tetrahydro-isoquinoline,

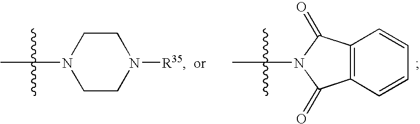

R³⁵ represents R¹⁰, SO₂—R¹⁰, COR¹⁰, or CONHR¹⁰;

E represents a bond, S(O)₀₋₂, or NR¹⁰;

Q, Q¹, Q² Q³, L¹ L², L³ and L⁴ independently at each occurance represent N-natural or unnatural amino acid side chain, CHR¹⁰, O, NH, S(O)₀₋₂, N—C(O)—NHR¹⁰, SO₂—N(R¹⁰)₂ N—C(O)—NH— (CH₂)₁₋₄—R²⁶, NR¹⁰, N-heteroaryl, N—C(═NH)—NHR¹⁰, or N—C (═NH)C₁₋₄ alkyl;

R²⁶ represents OH, NH₂, or SH;

R⁴¹ represents NH₂, NHR¹⁰, or N(R¹⁰), NHNH₂, NHOH, NR¹⁰NH₂, NHN(R¹⁰)₂, NH(C═NH)NH₂, NH(C═O)N(R¹⁰)₂;

provided that, (i) not all of X¹, X², X³ and X⁴ represent N or N—O simultaneously.

### DETAILED DESCRIPTION OF THE INVENTION

Preferred embodiments of the present invention provide compounds of Formula I, wherein

R¹ represents OH, halogen or COOH;

R², R³, R⁴, and R⁵ independently at each occurance represent H, SH, OR¹⁰, halogen, COOR¹⁰, (CH₂)₀₋₄—CONR¹¹R¹², optionally substituted aryl, optionally substituted heterocyclyl, C₄₋₁₄ cycloalkyl-C₁₋₄ alkyl, C₁₋₄ alkyl aryl, optionally substituted C₁₋₁₄ straight chain, branched or cyclo alkyl, O—(CH₂)₂₋₆—NR¹⁰—(CH₂)₀₋₃—R²⁴, NR¹⁰R²⁴, (CH₂)₁₋₄—NR³³R³⁴, (CH₂)₁₋₄—COOR³³, O—(CH₂)₁₋₃—CO—het, O— (CH₂)₁₋₂—NH—CO-aryl, O—(CH₂)₁₋₂-NR¹⁰—CO—NR¹⁰R³³, O—(CH₂)₀₋₂—C(O)—NR³³R³⁴, O—(CH₂)₁₋₄-COOR¹⁰, O—(CH₂)₁₋₃-het-R³², O-optionally substituted cycloalkyl, O—(CH₂)₁₋₄—NR¹⁰—COO-t-butyl, O—(CH₂)₁₋₄—NR¹⁰R³³, O—(CH₂)₁₋₄—NR¹⁰—C(O)—C₀₋₃-alkyl-optionally substituted aryl, O-substituted cycloalkyl, O—(CH₂)₀₋₆-optionally substituted aryl, (CH₂)₁₋₄—NH—C(O)O—(CH₂)₁₋₄-PhR¹³R¹⁴, NO₂, O—(CH₂)₀₋₄—C(O)—NH-tetrahydro carboline, NR¹⁰R²⁸, O—(CH₂)₁₋₃—optionally substituted het, CH₂COOCH₃, CH═CH—COOCH₃, 5-amidino benzimidazole,

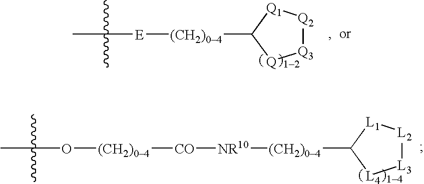

alternatively R² and R³ taken together form

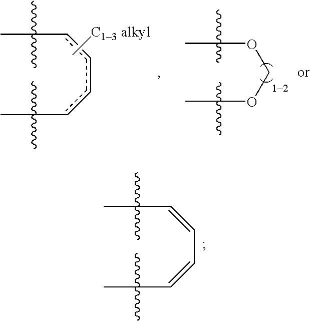

X¹ represents C—R⁶, N or N—O;

X² represents C—R⁷;

X³ represents C—R⁸;

X⁴ represents C—R⁹;

Z¹ represents C;

Z² represents N;

R⁶, R⁸ and R⁹ independently at each occurance represents H, halogen, cyano, C₁₋₄ alkyl, C₁₋₄ halogenated alkyl, NO₂, O-aryl or OR¹¹;

R⁷ represents NH₂, NHR¹⁰, N(R¹⁰)₂, NHSO₂—C₁₋₁₄ alkyl, NHSO-aryl, OH, NHCO—C₁₋₁₄ alkyl, NHNH₂, NHOH, NHCO—C₁₋₁₄ alkyl, NR¹⁰NH₂, NHN(R¹⁰)₂, NH(C═NH)NH₂, NH(C═O)N(R¹⁰)₂; alternatively

R⁶ and R⁷, R⁷ and R⁸, R⁸ and R⁹, along with the respective carbon atoms to which they are attached, can be taken together to represent a 6 saturated or aromatic, carbocyclic or heterocyclic ring structure substituted with R⁴¹;

R²⁰ represents R²⁴, C₁₋₄-alkyl, (CH₂)₁₋₃-biphenyl, (CH₂)₁₋₄-Ph-N(SO₂—C₁₋₂-alkyl)₂, (CH₂)₁₋₄—NH—C(O)—R²⁴, (CH₂)₁₋₄—NH—SO₂—R²⁴, halogen, COOR¹⁰, (CH₂)₁₋₄-Ph-N(SO₂—C₁₋₂alkyl), (CH₂)₁₋₄—NR¹⁰—C(O)—R²⁴, (CH₂)₁₋₄—NR¹⁰—SO₂—R²⁴, (CH₂)₁₋₄-het, (CH₂)₁₋₄—CON(R¹⁰)₂, (CH₂)₁₋₄—N(R¹⁰)—C(O)—NR¹⁰R²⁴, (CH₂)₁₋₄—N(R¹⁰)—C(S)—NR¹⁰R²⁴, or (CH₂)₁₋₃—COOH;

R²⁴ represents R¹⁰, (CH₂)₁₋₄-optionally substituted aryl, (CH₂)₀₋₄OR¹⁰, CO— (CH₂)₁₋₂—N(R¹⁰)₂, CO(CH₂)₁₋₄—OR¹⁰, (CH₂)₁₋₄—COOR¹⁰, (CH₂)₀₋₄—N(R¹⁰)₂, SO₂R¹⁰, COR¹⁰, CON(R¹⁰)₂, (CH₂)₀₋₄-aryl-COOR¹⁰, (CH₂)₀₋₄-aryl-N(R¹⁰)₂, or (CH₂)₁₋₄-het-aryl;

R²⁸ represents (CH₂)₁₋₂-Ph-O—(CH₂)₀₋₂-het-R³⁰, C(O)-het, CH₂-Ph-CH₂-het-(R³⁰)₁₋₃; (CH₂)₁₋₂-cyclohexyl-R³¹, CH₂-Ph-O-Ph-(R³⁰)₁₋₂, CH₂—(CH₂OH)-het-R³⁰, CH₂-Ph-O-cycloalkyl-R³¹, CH₂-het-C(O)—CH₂-het-R³, or CH₂-Ph-O—(CH₂) —O-het-R³⁰;

R³⁰ represents SO₂N(R¹⁰)₂, H, NHOH, amidino, or C(═NH)CH₃;

R³¹ represents R³⁰, amino-amidino, NH—C(═NH)CH₃ or R¹⁰;

R³² represents H, C(O)—CH₂—NH₂, or C(O)—CH(CH(CH₃)₂)—NH₂;

R³³ and R³⁴ independently at each occurance represent R¹⁰, (CH₂)₀₋₄—Ar, optionally substituted aryl, (CH₂)₀₋₄ optionally substituted heteroaryl, (CH₂)₁₋₄—CN, (CH₂)₁₋₄—N(R¹⁰)₂, (CH₂)₁₋₄—OH, (CH₂)₁₋₄—SO₂—N(R¹⁰)₂;

alternatively, R³³ and R³⁴ along with the nitrogen atom that they are attached to forms a 4 to 14 atom ring structure selected from tetrahydro-1H-carboline; 6, 7-Dialkoxyoxy-2-substituted 1,2,3,4-tetrahydro-isoquinoline,

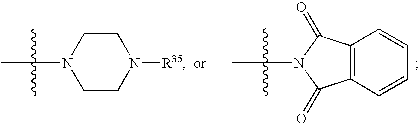

R³ represents R¹⁰ SO₂—R¹⁰, COR¹⁰, or CONHR¹⁰;

E represents a bond, S(O)₀₋₂, or NR¹⁰;

W₁, W₂, W₃ and W₄ independently represent C or N; and

Q, Q¹, Q², Q³, L¹, L², L³ and L⁴ independently at each occurance represent N-natural or unnatural amino acid side chain, CHR¹⁰, O, NH, S(O)₀₋₂, N—C(O)—NHR¹⁰, SO₂—N(R¹⁰)₂, N—C(O)—NH— (CH₂)₁₋₄—R²⁶, NR¹⁰, N-heteroaryl, N—C(═NH)—NHR¹⁰, or N—C(═NH)C₁₋₄ alkyl;

R represents OH, NH₂, or SH; and

provided that, (i) not all of X¹, X², X³ and X⁴ represent N or N—O simultaneously.

Provided in yet another preferred embodiment is a compound of Formula I, wherein

R¹ represents OH, O-Ph, COOH, or P(O) (OH)₂;

R² represents H, halo, optionally substituted alkyl or optionally substituted aryl or heteroaryl;

R³ represents C₀₋₆ alkyl-COOH;

R⁵ represents H, C₁₋₄ alkyl or OR¹⁰;

X¹ represents N or N—O;

R⁷ represents NH₂ or NHC₁₋₃ alkyl;

R²⁰ represents H, C₁₋₂ alkyl, (CH₂)₁₋₄-optionally substituted aryl, (CH₂)₁₋₄-het; (CH₂)₁₋₄—N(R¹⁰)₂, (CH₂)₁₋₄—CON(R¹⁰)₂, (CH₂)₁₋₄NR¹⁰—C(O)—R²⁴, (CH₂)₁₋₄—NR¹⁰—SO₂—R²⁴, or (CH₂)₁₋₃—COOH.

Another embodiment of the present invention provides compounds of Formula I wherein, X¹ represents C—R⁶; X² represents C—R⁷; X³ represents N or N—O; X⁴ represents C—R⁹; Z¹ represents C; and Z² represents N. Further preferred compounds are those wherein, R¹ represents OH, COOH, or P(O) (OH)₂; R² represents H, halo, optionally substituted alkyl or optionally substituted aryl or heteroaryl; R³ represents C₀₋₆ alkyl-COOH; R represents H, C₁₋₄ alkyl or OR¹⁰; X¹ represents N or N—O; R⁷ represents NH₂ or NHC₁₋₃ alkyl; R²⁰ represents H, C₁₋₂ alkyl, (CH₂)₁₋₄-optionally substituted aryl, (CH₂)₁₋₄-het; (CH₂)₁₋₄—N(R¹⁰)₂, (CH₂)₁₋₄—CON(R¹⁰)₂, (CH₂)₁₋₄—NR¹⁰—C(O)—R²⁴, (CH₂)₁₋₄—SO₂—R²⁴, or (CH₂)₁₋₃—COOH.

Further preferred compounds of Formula I are those wherein, R¹ represents OH or COOH; R⁴ represents (CH₂)₀₋₆—COOR¹⁰, optionally substituted heteroaryl, (CH₂)₀₋₄—CONR¹⁰R¹¹, C₁₋₁₀-straight chain alkyl, branched alkyl or cycloalkyl group substituted with 1-3 groups selected from COOR¹⁰, CONHR¹⁰, OR¹⁰, or aryl; and R represents NH₂. Yet further preferred compounds of Formula I are those wherein R¹ represents OH;

R² represents H, halogen, OH, phenyl, heteroaryl or substituted phenyl; R⁴ represents H, halo, (CH₂)₀₋₄-COOR¹⁰, (CH₂)₀₋₄—CONH₂, (CH₂)₀₋₄—CONHR³³, (CH₂)₀₋₄-heteroaryl, C₁₋₈ branched alkylene-COOR¹⁰, or C₂₋₆ alkenelyne-COOR¹⁰; and R²⁰ represents H or (CH₂)₀₋₃-optionally substituted phenyl, (CH₂)₀₋₃-aryl or (CH₂)₀₋₃-heteroaryl.

Specifically preferred compounds of Formula i provided by the present invention are:

[3-(5-Amino-3-benzyl-1H-pyrrolo[3,2-b]pyridin-2-yl)-5-chloro-4-hydroxy-phenyl]-acetic acid ethyl ester;

8-(5-Amino-3-benzyl-1H-pyrrolo[3,2-b]pyridin-2-yl)-6-bromo-7-hydroxy-3,4-dihydro-2H-naphthalen-1-one;

3-[3-(5-Amino-3-benzyl-1H-pyrrolo[3,2-b]pyridin-2-yl)-5-chloro-4-hydroxy-phenyl]-propionic acid;

[5-(5-Amino-3-benzyl-1H-pyrrolo[3,2-b]pyridin-2-yl)-6-hydroxy-3′-nitro-biphenyl-3-yl]-acetic acid;

[3-(5-Amino-3-benzyl-1H-pyrrolo[3,2-b]pyridin-2-yl)-5-chloro-4-hydroxy-phenyl]-acetic acid;

2-[3-(5-Amino-3-benzyl-1H-pyrrolo[3,2-b]pyridin-2-yl)-5-chloro-4-hydroxy-phenyl]-acetamide;

2-[3-(5-Amino-3-benzyl-1H-pyrrolo[3,2-b]pyridin-2-yl)-5-chloro-4-hydroxy-phenyl]-N-(2-morpholin-4-yl-ethyl)-acetamide;

2-(5-Amino-1H-pyrrolo[2,3-c]pyridin-2-yl)-4,6-dichloro-phenol;

8-(5-Amino-3-benzyl-1H-pyrrolo[3,2-b]pyridin-2-yl)-6-bromo-7-hydroxy-3,4-dihydro-2H-naphthalen -1-one;

3-[3-(5-Amino-3-benzyl-1H-pyrrolo[3,2-b]pyridin-2-yl)-5-chloro-4-hydroxy-phenyl]-propionic acid;

[5-(5-Amino-3-benzyl-1H-pyrrolo[3,2-b]pyridin-2-yl)-6-hydroxy-3′-nitro-biphenyl-3-yl]-acetic acid;

[3-(5-Amino-1H-pyrrolo[3,2-b]pyridin-2-yl)-5-chloro-4-hydroxy-phenyl]-acetic acid;

[5-(5-Amino-1H-pyrrolo[3,2-b]pyridin-2-yl)-6-hydroxy-31-nitro-biphenyl-3-yl]-acetic acid;

2-[5-(5-Amino-1H-pyrrolo[3,2-b]pyridin-2-yl)-6-hydroxy-31-nitro-biphenyl-3-yl]-N-(2-morpholin-4-yl-ethyl)-acetamide;

2-[5-(5-Amino-1H-pyrrolo[3,2-b]pyridin-2-yl)-6-hydroxy-3′-nitro-biphenyl-3-yl]-N-(2-methoxy-et hyl)-acetamide; and

2-[5-(5-Amino-1H-pyrrolo[3,2-b]pyridin-2-yl)-6-hydroxy-3′-nitro-biphenyl-3-yl]-N-(2-dimethylamino-ethyl)-acetamide.

Provided in yet another aspect of the present invention is a pharmaceutical composition comprising a pharmaceutically acceptable carrier and a therapeutically effective amount of a compound of Formula I or a pharmaceutically acceptable salt thereof. Yet another aspect of the present invention provides a method for treating or preventing a thromboembolic disorder, comprising administering to a patient in need thereof a therapeutically effective amount of a compound according to claim 2 or a pharmaceutically acceptable salt thereof.

### EXPERIMENTAL

Novel compounds of the present invention can be prepared in a number of ways known to one skilled in the art of organic synthesis. Described herein are some of the preferred synthetic methods for synthesizing novel compounds of the present invention. All temperatures reported herein are in degrees Celsius, unless indicated otherwise.

The novel compounds of Formula I can be prepared using the reactions and synthetic techniques described below. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

Proton NMR's (¹H NMR) were obtained using deuterated solvents such as dimethyl sulfoxide (DMSO-d₆), deuterated chloroform (CDCl₃), or other appropriate solvents. Compounds of the present invention can be prepared by synthetic schemes outlined below:

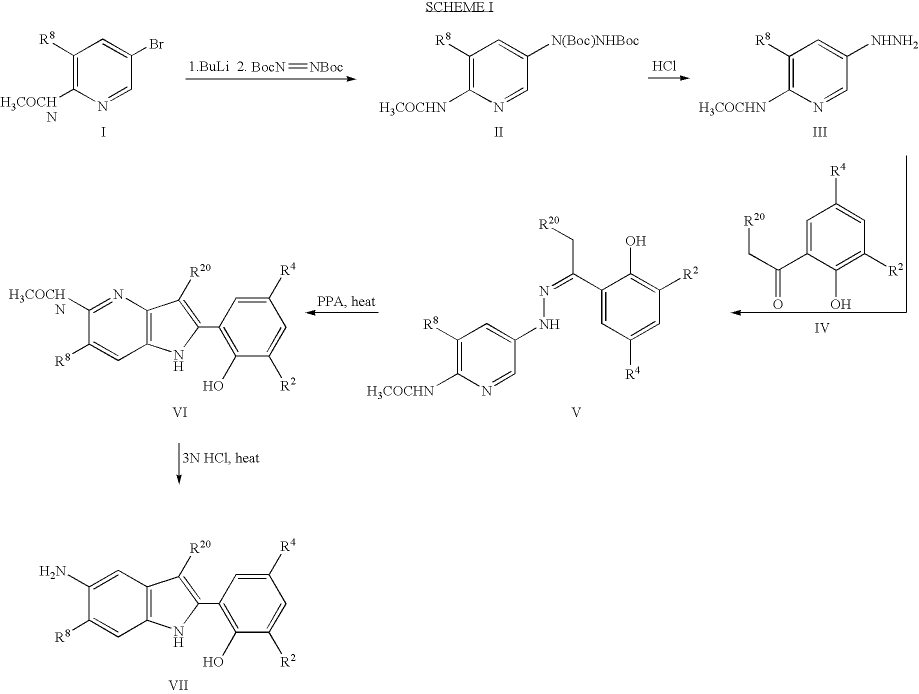

The following discussion provides the experimental details for the synthetic Scheme I above:

N-(6-Acetylamino-pyridin-3-yl)-N′-(2,2-dimethyl -propionyl) -hydrazinecarboxylic Acid Tert-Butyl Ester (II).

A mixture of N-(5-bromo-pyridin-2-yl)acetamide (I) (15.05 g, 70 mM) and THF (150 mL) was mixed with a solution of BuLi in hexanes (2.5 M, 70 mL, 175 mM) at −78° C. The resulting solution was agitated for 10 min at −78° C. A mixture of di-tert-butylazodicarboxylate (20.1 g, 87.5 mM) and THF (40 mL) then was added, and the reaction mixture was agitated for 25-30 min at about −78° C. The reaction mixture was warmed to ambient temperature and agitated for about 1 h. The mixture was combined with ice, the pH was adjusted to about 7 with 1N HCl, and the cold reaction mixture was washed with ether (×2). The combined ether extracts were dried (MgSO₄), filtered and concentrated under reduced pressure to form a residue. The residue was purified by column chromatography on silica gel using ethyl acetate/hexanes as the eluent (30:70) to afford 5.9 g (24%) of II as an oil.

NMR-¹H (CDCl₃) δ: 1.47 (s, 18H), 2.20 (s, 3H), 7.55 (br. s, 1H), 7.79 (d, J=8.8 Hz, 1H), 8.17 (d, J=8.8 Hz, 1H), 8.32 (br s, 1H), 8.64 (s, 1H).

N-(5-Hydrazino-pyridin-2-yl)-acetamide Dihydrochloride (III)

A mixture of II (5.2 g, 14.2 mM) and DCM (15 mL) was mixed with 4N HCl in dioxane (15 mL) and the resulting reaction mixture was let stand for about 18 h to form a precipitate. The precipitate was isolated, washed sequentially with (DCM and ether) and dried under reduced pressure (vacuum) to afford 3.8 g (98%) of III as a white powder.

MS (MH+): found: 167.0; calc.: 166.09.

(3-{1-[(6-Acetylamino-pyridin-3-yl)-hydrazono]-3-phenyl-propyl}-5-chloro-4-hydroxyphenyl-acetic Acid (V).

A mixture of III (1.3 g, 5 mM), [3-chloro-4-hydroxy-5-(3-phenyl-propionyl)-phenyl]-acetic acid (IV) (1.0 g, 3.3 mM) and ethanol (15 mL) was diluted with triethylamine to adjust the pH to about 9.5. The resulting mixture was refluxed for 2.5-3 h and the solvent was removed under reduced pressure to yield a residue. The residue was treated with 5% aqueous citric acid to form a precipitate. The resulting precipitate was filtered, washed with H₂O and dried in a vacuum oven over P₂O, to afford 2.0 g (98%) of V as a white solid.

MS (MH+): found: 468; calc. 466.14.

[3-(5-Acetylamino-3-benzyl-1H-pyrrolo[3,2-b]pyridin-2-yl)-5-chloro-4-hydroxy-phenyl]-acetic Acid (VI).

A mixture of (V) (0.8 g, 1.88 mM) and a polyphosphoric acid (˜6 mL) was heated at 125° C. for 45-60 min. The reaction mixture was cooled and the resulting suspension was neutralized with 50% NaOH, while maintaining the temperature of the reaction mixture at or below ambient temperature, to form a precipitate. The precipitate was isolated, washed with water, and purified by reverse phase HPLC (acetonitrile/0.02 N HCl gradient) to give 90 mg (12%) of VI as an off-white solid.

NMR-¹H (DMSO-d,) δ: 2.15 (s, 3H), 3.60 (s, 2H), 4.12 (s, 2H), 6.91-7.25 (m, 7H), 7.41 (s, 1H), 7.54 (br. s, 1H), 8.03 (br s, 1H), 9.75 (br. s, 1H).

MS (MH+): found: 450.0; calc.: 449.11.

Ex. 1: [3-(5-Amino-3-benzyl-1H-pyrrolo[3,2-b]pyridin-2-yl)-5-chloro-4-hydroxy-phenyl]-acetic Acid Hydrochloride (VI1, R²═CH₂COOH)

A mixture of VI (88 mg, 0.196 mM) and 3N HCl was refluxed for 45-130 min. The solvent was evaporated under reduced pressure to yield a residue. The residue was washed with cold water and dried over P₂O₅ under reduced pressure (vacuum) to yield 86 mg (98%) of VII as an off-white solid.

NMR-¹H (DMSO-d₆) δ: 3.54 (s, 2H), 4.03 (s, 2H), 6.63 (d, J=9.2 Hz, 1H), 6.79-7.27 (m, 6H), 7.35 (s, 1H), 7.51 (br. s, 1H), 7.98 (d, J=9.2, 1H), 9.65 (br s, 1H), 12.06 (s, 1H), 12.29 (br s, 1H), 13.81 (br s, 1H).

MS (MH+): found: 408.1; calc.: 407.10.

Ex. 2: 3-[3-(5-Amino-3-benzyl-1H-pyrrolo[3,2-b]pyridin-2-yl)-5-chloro-4-hydroxy-phenyl]-propionic Acid Hydrochloride (VII, R²═CH₂CH₂COOH)

This compound was prepared using the procedure in X above.

NMR-¹H (DMSO-d₆) δ: 2.39 (t, J=7.X , 2H), 2.63 (t, J=7.6 Hz, 2H), 4.05 (s, 2H), 6.63 (d, J=9.2 Hz, 1H), 6.92 (s, 1H), 7.00-7.18 (m, 5H), 7.31 (s, 1H), 7.46 (br s, 1H), 7.98 (d, J=9.2 Hz, 1H), 9.55 (br s, 1H), 12.03 (s, 1H), 13.67 (br s, 1H).

MS (MH+): found: 421.9; calc.: 421.12.

Ex. 3:[5-(5-Amino-3-benzyl-1H-pyrrolo[3,2-b]pyridin-2-yl)-6-hydroxy-3′-nitro-biphenyl-3-yl]-acetic Acid Hydrochloride (VII, R¹=3-nitrophenyl).

MS (MH+): found: 495.2; calc.: 494.16.

Ex. 21

NMR-¹H (DMSO-d₆) δ: 2.22 (s, 3H), 3.44 (s, 2H), 4.05 (s, 2H), 6.76-7.23 (m, 6H), 7.7.25-7.57 (m, 2H), 7.85 (s, 1H), 9.60 (s, 1H), 11.96 (s, 1H).

MS: found (MH+) 421.9, calc. 421.12

Ex. 22

MS: found (M+H) 436.0, calc 437.04

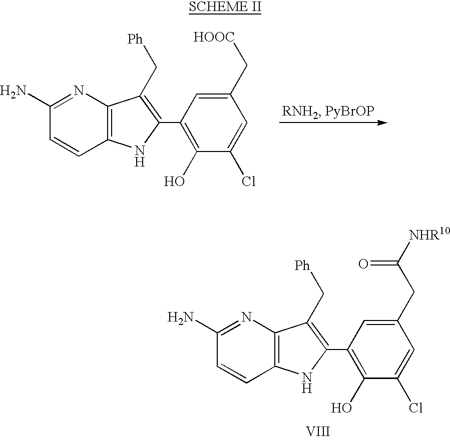

Ex. 15: 2-[3-(5-Amino-3-benzyl-1H-indol-2-yl)-5-chloro-4-hydroxy-phenyl]-acetamide Hydrochloride (VIII, R¹⁰═H)

This compound was prepared by treating the corresponding carboxylic acid with an excess of ammonia solution and PyBrOP, with DMF as the reaction medium, followed by purification on a reverse phase HPLC column using acetonitrile/0.02N HCl gradient.

MS (MH+): found: 407.2; calc.: 406.12.

Ex. 16: 2-[3-(5-Amino-3-benzyl-1H-indol-2-yl)-5-chloro-4-hydroxy-phenyl]-N-(2-morpholin-4-yl-ethyl) -acetamide dihydrochloride (VIII, R═CH₂CH₂—N-morpholinyl).

MS (MH+): found:520.3; calc.: 519.2.

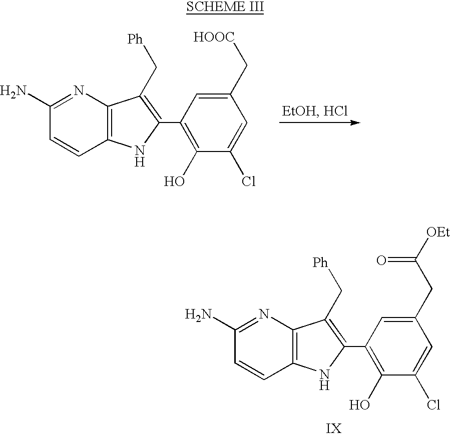

Ex.: 18: [3-(5-Amino-3-benzyl-1H-pyrrolo[3,2-b]pyridin-2-yl)-5-chloro-4-hydroxy-phenyl]-acetic Acid Ethyl Ester Hydrochloride (IX)

This compound was prepared by heating the solution of the corresponding carboxylic acid in ethanol with 3N HCl in dioxane. After evaporation of the solvents the residue was redissolved in 5% acetonitrile in water and lyophilized.

MS (MH+): found: 436.1; calc.: 435.13.

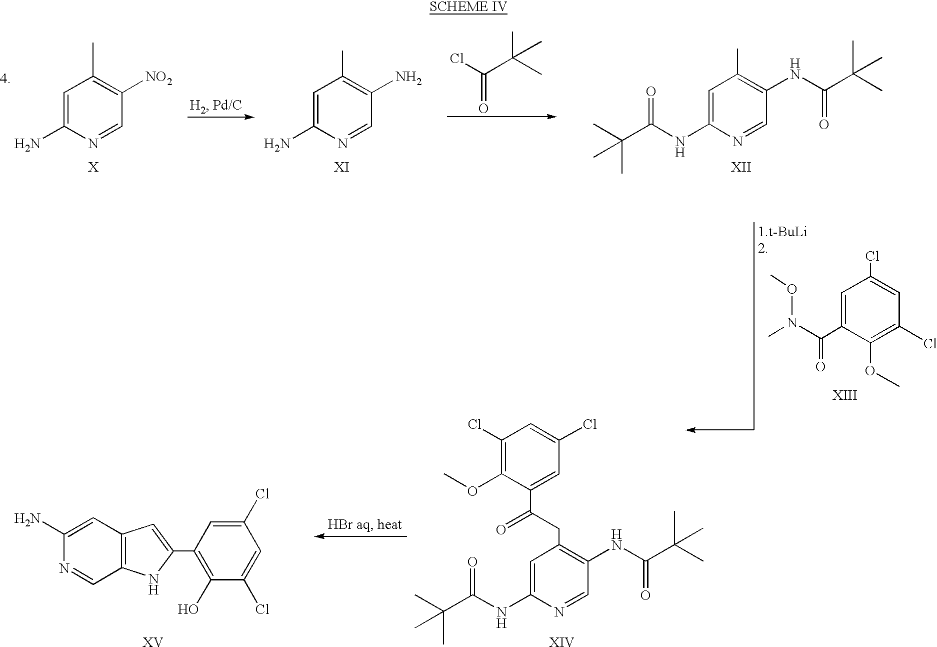

2,5-Diamino-4-methylpyridine (XI)

A heterogeneous mixture of 2-amino-4-methyl-5-nitropyridin (X) (0.83 g, 5,4 mM), 10% Pd/C (0.24 g) and THF (15 mL) was agitated under 50 psi of hydrogen for 3 h. The reaction mixture was filtered through celite, and concentrated under reduced pressure to yield XI as an off white crystalline solid.

N-[6-(2,2-Dimethyl-propionylamino)-4-methyl-pyridin-3-yl]-2,2-dimethyl-propionamide (XII)

A mixture of XI (0.66 g, 5.4 mM), from above, triethylamine (1.2 g, 12 mM) and THF (25 mL) was agitated at 5-10° C. The agitated mixture then was combined with trimehtylacetyl chloride 91.46 g, 11.9 mM) and a catalytic amount of DMAP. The resulting mixture was agitated for 8-16 h, diluted with a 5% solution of citric acid, and extracted with ethyl acetate. The combined organic extracts were sequentially washed with water and brine, dried (MgSO₄), filtered and the filtrate was concentrated under reduced pressure to yield a residue. The residue was purified by column chromatography on silica gel with ethyl acetate/hexanes as the eluent (25:75) to afford 0.95 g (60%) of XII as a white powder.

NMR-¹H (CDCl₃) δ: 1.30 (s, 9H), 1.33 (s, 9H), 2.23 (s, 3H), 7.15 (s, 1H), 7.99 (s, 1H), 8.15 (s, 1H), 8.47 (s, 1H).

3,5-Dichloro-2,N-dimethoxy-N-methyl-benzamide (XIII)

A mixture of 3,5-dichloro-2-methoxy benzoic acid (1.7 g, 7.17 mM), N,O-dimethylhydroxylamine (0.9 g, 9 mM), PyBrOP (4.4 g, 9.3 mM), HOBt (1.25 g, 9.3 mM), triethylamine (2.9 g, 28.8 mM) and DMF (25 mL) was agitated for 4 h at ambient temperature. This reaction mixture then was diluted with water and extracted with a mixture of ethyl ether/ethyl acetate. The combined extracts were sequentially washed with 5% aqueous sodium bicarbonate (2×), H₂O, and brine. The organic layer was dried (MgSO₄), filtered and concentrated under reduced pressure to yield a residue. The residue was purified by column chromatography on silica gel with hexane/ethyl acetate 4:1 as the eluent to afford 1.1 g (61%) of XIII as a white solid.

MS (MH+): found: 263.8; calc.: 263.0.

N-[4-[2-(3,5-Dichloro-2-methoxy-phenyl)-2-oxo-ethyl]-6-(2,2-dimethyl-propionylamino)-pyridin-3-yl]-2,2-dimethyl-propionamide (XIV)

A mixture of XII (0.78 g, 2.7 mM) and THF (10 mL) was cooled to about −40° C. This cold mixture was combined with t-BuLi in hexanes (1.7M, 6.2 mL, 10.5 mM). The resulting mixture was agitated at about −40° C. for 1 h. This agitated mixture then was combined with a THF solution of XIII (1.06 g, 4.0 mM). This mixture was let stand at about −40° C. for 3 h, warmed to ambient temperature and let stand at ambient temperature for 15-20 h. The reaction mixture then was mixed with an aqueous 5% citric acid mixture and extracted with ethyl acetate. The combined organic layers were washed with water and brine, dried (MgSO₄) and concentrated under reduced pressure to yield a residue. The residue was recrystallized from ethyl acetate-hexane to afford 0.68 g (51%) of XIV as a white solid.

MS (MH+): found: 494.1; calc.: 493.15.

Ex. 101: 2-(5-Amino-1H-pyrrolo[2,3-c]pyridin-2-yl)-4,6-dichlorophenol Hydrochloride (XV)

A solution of XIV (0.2 g 0.4 mM) in 33% aqueous HBr (10 mL) was refluxed for 6 h. After cooling a yellow precipitate was filtered, washed with water, 5% solution of sodium bicarbonate , water and dried in a high vacuum over phosphorus pentoxide to give 0.086 g (72%) of XV as a yellow powder. The material was further purified by reverse phase HPLC (acetonitrile/0.02 N HCl gradient) to generate the HCl salt of XV.

NMR-¹H (DMSO-d₆) δ: 6.62 (br.s, 2H), 6.92 (s, 1H), 7.04 (s, 1H), 7.69 (d, J=2.2 Hz, 1H), 7.85 (d, J=2.2 Hz, 1H), 8.32 (s, 1H), 10.66 (br.s, 1H), 12.00 (s, 1H), 13.01 (br s, 1H).

MS (MH+): found: 293.9; calc.; 293.01.

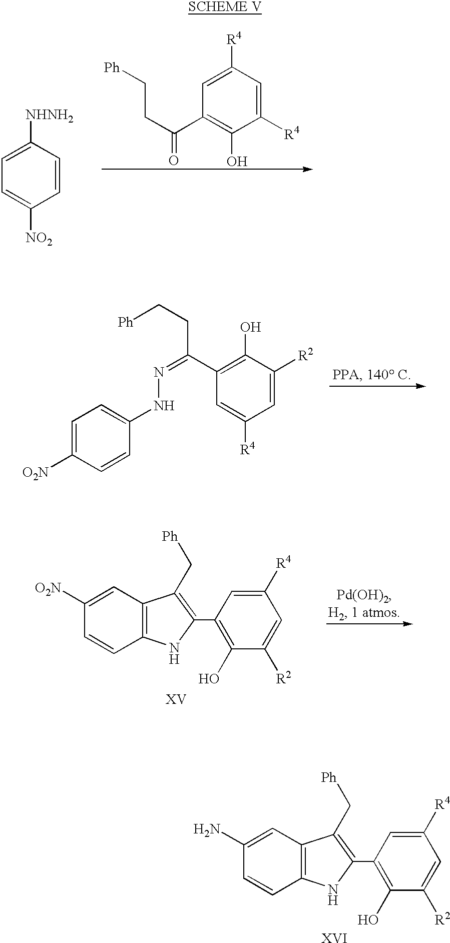

[3-(3-Benzyl-5-nitro-1H-indol-2-yl)-5-chloro-4-hydroxy-phenyl]-acetic Acid (XV)

This compound was prepared by using the procedure to synthesize compound VI in Scheme I abovce. The compound was purified by reverse phase HPLC (acetonitrile/0.02N HCl gradient).

MS: found (MH−) 435.0, calc 436.08.

Ex. 201: [3-(5-Amino-3-benzyl-1H-indol-2-yl)-5-chloro-4-hydroxy-phenyl]-acetic acid hydrochloride (XVI)

This compound was prepared by reducing the corresponding nitro precursor by using the procedure to make compound XI in Scheme IV above. Purification by reverse phase HPLC (acetonitrile/0.02N HCl gradient) yielded the title compound.

MS (MH+): found: 407.2; calc.: 406.11.

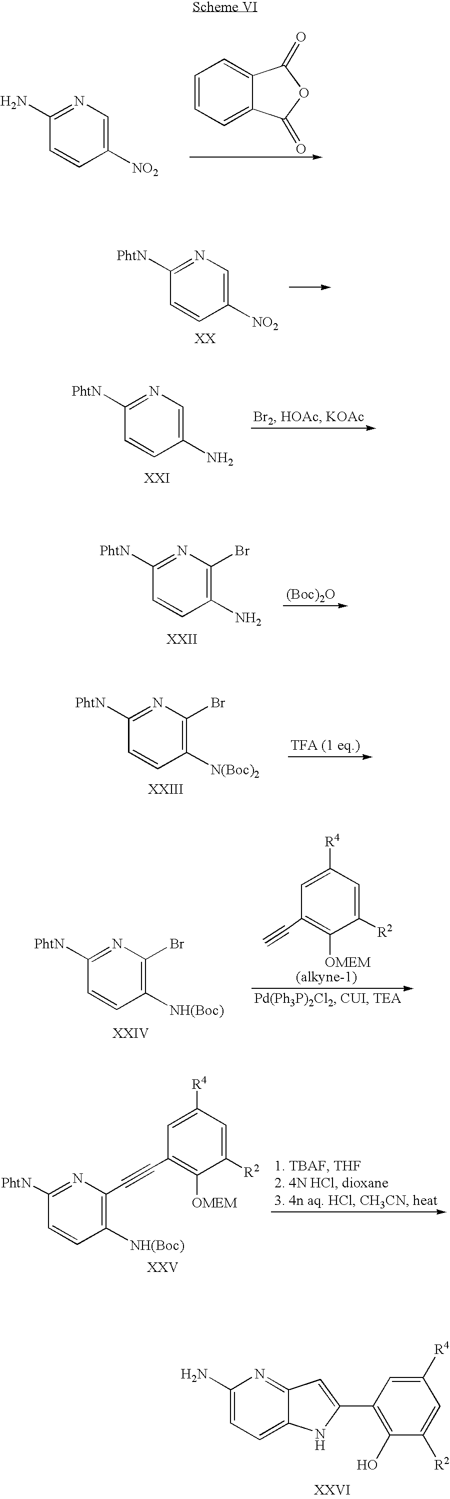

2-(5-Nitro-pyridin-2-yl)-isoindole-1,3-dione (XX)

2-amino-5-nitropyridine (30.00 g, 215.6 mol), phthalic anhydride (35.50 g, 239.7 mol) and DMF (10 mL) were heated at about 210° C. in a sand bath for about 3 h. The reaction mixture was cooled to ambient temperature and let stand for about 2 h leading to the formation of crystals. The crystals were isolated and washed with ethanol (×2) to yield the compound of formula XX. (53.0 g, 91%).

MS: found (MH+) 270.2, calc 269.21.

2-(5-Amino-pyrdin-2-yl)-isoindole-1,3-dione (XXI)

A mixture of con. HCl (500 mL) and XX (20.00 g, 74.3 mmol) was cooled to about 70° C. A solution of tin (II) chloride dihydrate (50.35 g, 223.2 mmol) and conc. HCl (60 mL) was mixed with a solution of XX and HCl at about 0° C., and resulting solution was allowed to warm to ambient temperature. The reaction mixture then was mixed with 600 mL water and the resulting reaction mixture was agitated for about 10 minutes to form a bright yellow reaction mixture. This reaction mixture was washed with 4n HCl (×1) and the washed reaction mixture was concentrated under reduced pressure to yield a compound of formula XXI (26.87 g, >100%).

MS: found (MH+) 240.2, calc 239.23

2-(5-Amino-6-bromo-pyridin-2-yl)-isoindole-1,3-dione (XXII)

A solution of XXI (crude 26.87 g) in acetic acid (100 mL) was mixed with a solution of KOAc (7.3 g, 74.4 mmol) in acetic acid (100 mL). The resulting reaction mixture then was mixed with a solution of Br₂ (4.19 mL, 81.8 mmol) in acetic acid (50 mL) to form a new reaction mixture. The new reaction mixture then was agitated at ambient temperature for about 12-16 hours. The agitated reaction mixture then was diluted with water (600 mL) and the resulting mixture was agitated for about 10 minutes. The reaction solids were isolated, dried and dissolved in methylene chloride. The methylene chloride solution was filtered through celite and the filtered methylene chloride solution was concentrated under reduced pressure to yield the compound of formula XXII (18.14 g, 76%).

MS: found (MH+) 319.0, calc 318.13.

[2-Bromo-6-(1,3-dihydro-isoindol-2-yl)-pyridin-3-yl]-biscarbamic Acid Tert-Butyl Ester (XXIII)

A solution of XXII (9.90 g, 31.1 mmol) in THF (250 mL) was mixed with Et₃N (19.4 mL, 139 mmol), di-tert-butyl dicarbonate ((Boc)₂O) (21.72 g, 99.4 mmol), and DMAP (catalytic) to form a reaction mixture. The reaction mixture was agitated at ambinet temperature from about 8 to about 16 hours. The agitated reaction mixture then was mixed with 5% citric acid until the pH of the reaction mixture reached about 5. The pH adjusted reaction mixture then was extracted with ethyl acetate and the ethyl acetate layer was sequentially washed with water (×1) and brine (×1), dried (Na₂SO₄) and concentrated under reduced pressure to yield the compound of formula XXIII (13.4 g, 83%).

MS: found (MH+) 519.4, calc 518.36

[2-Bromo-6-(1,3-dihydro-isoindol-2-yl)-pyridin-3-yl]-carbamic Acid Tert-Butyl Ester (XXIV)

A solution of XXIII (4.73 g, 9.12 mmol) and CH₂Cl₂ (25 mL) was mixed with CF₃COOH (1.25 mL, 16.2 mmol) at about 0° C. The reaction mixture then was warmed to ambient temperature and agitated at ambient temperature for about 6 h. The agitated reaction mixture then was concentrated under reduced pressure to yield a residue. The residue was diluted with ethyl acetate and the pH of the reaction mixture was adjusted to about 5 using a 5% aqueous solution of citric acid. The organic layer was isolated, and sequentially washed with water (×1) and brine (×1), dried (MgSO₄) and then concentrated under reduced pressure to yield a residue. The residue was diluted with ethyl acetate and the ethyl acetete solution was cooled in a freezer for about 12 hours leading to the formation of crystals. The crystals were isolated, washed with ethyl acetate, and dried under reduced pressure to yield the compound of formula XXIV (2.80 g, 73%).

¹H-NMR (DMSO-d₆) δ: 8.13 (d, 1H), 7.92 (m, 2H), 7.57 (s, 1H), 1.48 (s, 9H).

MS: found (MH+) 419.4, calc 418.24.

Compound XXV

A mixture of alkyne-1 (0.47 g, 1.00 mmol) and dry acetonitrile was combined with the compound of formula XXIV 0.42 g (1.00 mmol) to form a mixture. The mixture then was mixed with Et₃N (10.0 mmol, 1.4 mL) and nitrogen gas was bubbled through the reaction mixture for a couple of minutes. The preceeding reaction mixture then was mixed with CuI (3.8 mg, 0.02 mmol) and dichlorobis(triphenylphosphine)-palladium (II) (14.0 mg, 0.02 mmol) to form a new reaction mixture. The new reaction mixture then was refluxed for about an hour. The refluxed reaction mixture was cooled to ambinet temperature and the cooled reaction mixture was quenched with with 5% citric acid/EtOAc leading to the formation of a solid. The solid was isolated and purified by column chromatography on 10 g of silica (EtOAc/hexane) to yield the compound of formula XXV (0.37 g (45%).

¹H NMR (CDCl₃) δ: 1.51 (s, 9H), 2.66 76 (d d, j=6 Hz, 18 Hz, 1H), 3.10-3.20 (m, 6H), 3.25-3.35 (m, 2H), 3.62 (s, 3H), 3.65 (s, 3H), 4.06 (d d, J=6 Hz, 11 Hz, 1H), 5.00 (s, 2H), 7.27 (d, J=3 Hz, 1H), 7.31 (d, J=9 Hz, 1H), 7.52 (d, J=3 Hz, 1H), 7.55 (t, J=7 Hz, 1H), 7.70-7.80 (m, 2H), 7.81 (d d, J -1 Hz, 7 Hz,), 7.87-7.93 (m, 2H), 8.17 (d of m, J=7 Hz, 1H), 8.39 (d, J=1 Hz, 1H), 8.70 (d, J=9 Hz, 1H).

Compound XXVI (Ex. 401) (R²=m-nitrophenyl, R⁴=4-(1,2-dicarboxy-ethyl)

A solution of the compound of formula XXIV (0.3 g, 0.38 mmol) and 3.0 mL of 1.0 M TBAF in THF was agitated at elevated temperatures (about 65° C.) for about 4 h. The raction mixture then was cooled to ambinet temperature, mixed with 5% citric acid/EtOAc and extracted with EtOAc. The EtOAc extracts were dreid (MgSO₄) and then concentrated under reduced pressure to yield crude pyrrolo[b]pyridine as a residue. The crude product then was dissolved in 2 ml dry methanol and 2 mL 4M HCl/dioxane. The resulting mixture was agitated for about 6 hours. The agitated reaction mixture was mixed with a sodium bicarbonate solution/EtOAc, the organic layer was isolated and washed with water. The aqueous layer was further acidified to a pH of about 2, and extracted with EtOAc. The organic layers were combined, dried 9 sodium sulfate) and concentrated under reduced pressure to yield a residue. The crude residue was further dissolved in acetonitrile (4 mL) and 4N HCl (4 mL) and the resulting mixture was heated to reflux for about 12 h. The reaction mixture was cooled to ambient temperature and concentrated under reduced pressure to yield a residue. The residue was purified using preparative HPLC to yield the compound of formula XXVI (64 mg, 34%).

¹H NMR (DMSO-d₆) δ: 2.52 (d d, J=6.2 Hz, 1H), 3.01 (m, 1H), 3.84 (m, 1H), 6.49 (d, J=9 Hz, 1H), 6.78 (s, 1H), 7.16 (d, J=2 Hz, 1H), 7.39 (br s, 1H), 7.57 (d, J=2 Hz), 7.65 (t, J=7 Hz, 1H), 7.86 (d, J=7 Hz, 1H), 7.91 (d, J=9 Hz, 1H), 8.11 (d, J=7 Hz, 1H), 8.24 (s, 1H).

MS: found (MH+) 463.3, calc 462.41.

The following examples were prepared using the procedure outlined in Scheme VI above.

Ex. 402

¹H NMR (DMSO-d₆) δ: 2.61 (m, 4H), 3.42 (virt. Quint, J=8.4 Hz, 1H), 6.56 (d, J=10.2 Hz, 1H), 6.86 (s, 1H), 7.24 (s, 1H), 7.46 (br. s , 2H), 7.63 (s, 1H), 7.69-7.75 (m, 1H), 7.91 (d, J=8.1 Hz, 1H), 8.00 (d, J-10.2 Hz, 1H), 8.18 (d, J=8.1 Hz, 1H), 8.33 (s,1H), 12.06 (br s, 1H).

MS found (MH+) 477.4, calc. 476.13.

Ex. 403:

¹H NMR (DMSO-d₆): 3.65 (s, 2H), 6.61 (d, J=8.8 Hz, 1H), 6.88 (s, 1H), 7.16 (s,1H), 7.61-7.37 (m, 6H), 8.05 (d, J=8.8 Hz, 1H), 9.06 (br s, 1H), 12.05 (br.s, 1H),

13.79 (br. s, 1H). MS; found (MH═) 360.0, calc. 359.13.

Ex. 404

¹H NMR (dmso-d₆) δ: 3.63 (s, 2H), 6.60 (d, J=9.1 Hz, 1H), 6.87 (s, 1H), 7.27 (s, 1H), 7.57 (br s, 1H), 7.67 (s, 1H), 7.76 (t, J=8.1 Hz, 1H), 7.96 (d, J=8.1 Hz, 1H), 8.04 (d, J=9.1 Hz, 1H), 8.23 (d, J=8.4 Hz, 1H), 8.36 (s, 1H), 9.62 (s, 1H), 12.43 (br.s, 2H), 14.07 (br. s, 1H).

MS: found (MH+) 405.1, calc 404.1.

Ex. 405:

¹H NMR (DMSO-d₆) δ: 6.66(d, J=7.7 Hz, 1H), 7.03 (s, 1H), 7.65 (s, 1H), 7.85 (br t, 1H), 8.11-8.05 (m, 3H), 8.30 (d, J=7.7 Hz, 1H), 8.47 (s, 1H). 8.54 (s, 1H), 12.35 (br.s, 1H), 13.95 (br.s, 1H).

MS: found (MH+) 414.3, (M−H) 413.0, calc 414.12.

Ex. 406

¹H NMR (DMSO-d₆): 2.56 (br t, 2H), 2.82 (br t, 2H), 6.62 (d, J=8.1 Hz, 1H), 6.91 (s, 1H), 7.27 (s, 1H), 7.57 (br s, 1H), 7.65 (t, J=7.7 Hz, 1H), 8.05 (d, J=8.1 Hz, 1H), 8.16 (d, J=9.1 Hz, 1H), 8.25 (d, J=7.7 Hz, 1H), 8.39 (s, 1H), 9.36 (s, 1H), 12.18 (br. s , 1H), 13.76 (br s, 1H).

MS: found (MH+) 419.3, calc 418.13.

Ex. 407:

¹H NMR (DMSO-d₆): 3.57 (s, 2H), 6.63 (d, J=9.1 Hz, 1H), 6.91 (s, 1H), 7.34 (s, 1H), 7.62 (br s, 3H), 8.03(d, J=9.1 Hz, 1H). MS: found (M−H) 315.8, calc 317.06.

Ex. 408

¹H NMR (DMSO-d₆): 2.68 (br. t, 2H), 2,92 (br t, 2H), 6.63 (d, J=8.8 Hz, 1H), 6.94 (s, 1H), 7.31 (s, 1H), 7.62 (br s, 3H), 8.04 (d, J-8.8 Hz, 1H), 10.00 (br s, 1H), 12.20 (br s, 1H), 13.97 (br s, 1H).

MS: found (MH+) 331.9, calc 331.07.

Ex. 409

MS: found (MH+) 366.4, calc 365.08

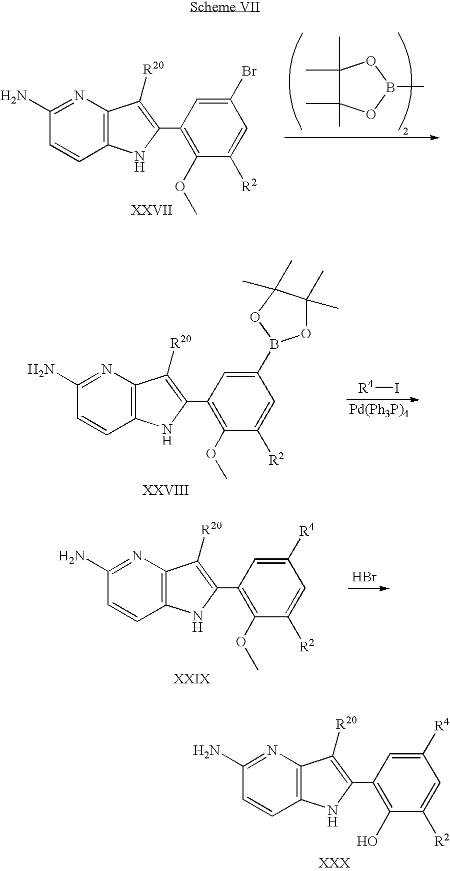

Tables I-VII list compounds that can be made using the synthetic schemes and procedures discussed above.

TABLE I

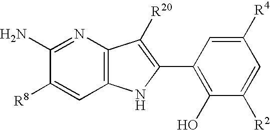

| Ex. | R² | R⁴ | R⁸ | R² |
|---|---|---|---|---|
| 1 | Cl | CH₂COOH | H | benzyl |
| 2 | Cl | CH₂CH₂COOH | H | benzyl |
| 3 | Cl | CH₂CONHR¹⁰ | H | benzyl |
| 4 | Ph | CH₂COOH | H | benzyl |
| 5 | Ph | CH₂CH₂COOH | H | benzyl |
| 6 | Br | CH₂CH₂COOH | H | benzyl |
| 7 | Br | CH₂COOH | H | benzyl |
| 8 | Cl | COOH | H | benzyl |
| 9 | Br | COOH | H | benzyl |
| 10 | 3-nitro- | CH₂COOH | H | benzyl |
| | phen-1-yl | | | |
| 11 | 3-nitro- | CH₂CH₂COOH | H | benzyl |
| | phen-1-yl | | | |
| 12 | Br | 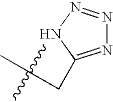 | H | benzyl |
| 13 | 3-nitro- | COOH | H | benzyl |
| | phen-1-yl | | | |
| 14 | Br | 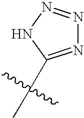 | H | benzyl |
| 15 | Cl | CH₂CONH₂ | H | benzyl |
| 16 | Cl | 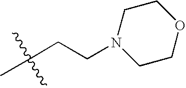 | H | benzyl |
| 17 | Cl | C(CH₃)COOH | H | benzyl |
| 18 | Cl | CH₂COOC₂H₅ | H | benzyl |
| 19 | Cl | CH═CH—COOH | H | benzyl |
| 20 | Cl | 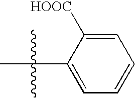 | H | benzyl |
| 21 | Br | CH₂COOH | CH₃ | benzyl |
| 22 | Br | COOH | H | benzyl |
| 23 | Cl | Br | H | benzyl |
| 24 | Cl | COOH | H | benzyl |
| 25 | Cl | 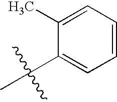 | H | benzyl |

TABLE II

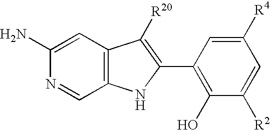

| Ex. | R² | R⁴ | R²⁰ |
|---|---|---|---|
| 101 | Cl | Cl | H |
| 102 | Cl | CH₂COOH | CH₂Ph |
| 103 | Ph | CH₂COOH | CH₂Ph |
| 104 | Br | CH₂COOH | CH₂Ph |
| 105 | Ph | CH₂CH₂COOH | CH₂Ph |
| 106 | Ph | CH₂CH₂COOH | CH₂Ph |
| 107 | Br | CH₂COOH | CH₂Ph |
| 108 | Cl | COOH | CH₂Ph |
| 109 | Br | COOH | CH₂Ph |
| 110 | 3-nitro- | CH₂COOH | CH₂Ph |
| | phen-1-yl | | |
| 111 | 3-nitro- | CH₂CH₂COOH | CH₂Ph |
| | phen-1-yl | | |
| 112 | Br | 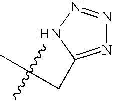 | CH₂Ph |
| 113 | 3-nitro- | COOH | CH₂Ph |
| | phen-1-yl | | |
| 114 | Br | 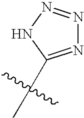 | CH₂Ph |
| 115 | Cl | CH₂CONH₂ | CH₂Ph |

TABLE III

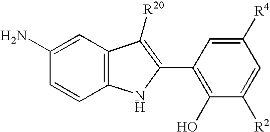

| Ex. | R² | R⁴ | R²⁰ |
|---|---|---|---|
| 201 | Cl | CH₂COOH | CH₂Ph |
| 202 | Cl | Cl | CH₂Ph |
| 203 | Ph | CH₂COOH | CH₂Ph |
| 204 | Br | CH₂COOH | CH₂Ph |
| 205 | Ph | CH₂CH₂COOH | CH₂Ph |
| 206 | Ph | CH₂CH₂COOH | CH₂Ph |

TABLE IV

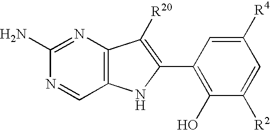

| Ex. | R² | R⁴ | R²⁰ |
|---|---|---|---|
| 301 | Cl | Cl | H |
| 302 | Cl | CH₂COOH | CH₂Ph |
| 303 | Ph | CH₂COOH | CH₂Ph |
| 304 | Br | CH₂COOH | CH₂Ph |
| 305 | Ph | CH₂CH₂COOH | CH₂Ph |
| 306 | Ph | CH₂CH₂COOH | CH₂Ph |
| 307 | Br | CH₂COOH | CH₂Ph |
| 308 | Cl | COOH | CH₂Ph |
| 309 | Br | COOH | CH₂Ph |
| 310 | 3-nitro- | CH₂COOH | CH₂Ph |
| | phen-1-yl | | |
| 311 | 3-nitro- | CH₂CH₂COOH | CH₂Ph |
| | phen-1-yl | | |
| 312 | Br | 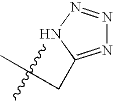 | CH₂Ph |
| 313 | 3-nitro- | COOH | CH₂Ph |
| | phen-1-yl | | |
| 314 | Br | 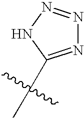 | CH₂Ph |
| 315 | Cl | CH₂CONH₂ | CH₂Ph |

TABLE V

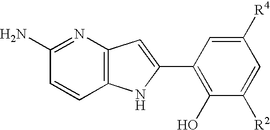

| Ex. | R² | R⁴ |
|---|---|---|
| 401 | m-nitrophenyl | 4-(1,2- |
| | | dicarboxy- |
| | | ethyl) |
| 402 | m-nitrophenyl | 4-(2-carboxy-1- |
| | | carboxymethyl- |
| | | ethyl) |
| 403 | phenyl | CH₂COOH |
| 404 | m-nitrophenyl | CH₂COOH |
| 405 | m-nitrophenyl | 2-tertazolyl |
| 406 | m-nitrophenyl | CH₂CH₂COOH |
| 407 | Cl | CH₂COOH |
| 408 | Cl | CH₂CH₂COOH |
| 409 | 3-thienyl | CH₂COOH |

TABLE VI

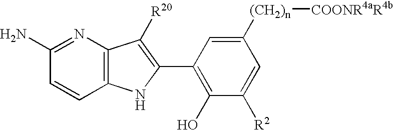

| Ex. | n | R² | R^{4a} | R^{4b} | R²⁰ |
|---|---|---|---|---|---|
| 501 | 1 | m-nitro | H | 2-morpholin- | H |
| | | phenyl | | 4-yl-ethyl | |
| 502 | 1 | Cl | 2-hydroxy | 2-hydroxy | benzyl |
| | | | ethyl | ethyl | |
| 503 | 1 | Cl | CH₂CH₂—SO₂—R^{4b} | CH₂CH₂ | benzyl |
| 504 | 1 | Cl | H | 2-methoxy | benzyl |
| | | | | ethyl | |
| 505 | 1 | m-nitro | H | 2-hydroxy | H |
| | | phenyl | | ethyl | |
| 506 | 1 | m-nitro | H | 2-methoxy | H |
| | | phenyl | | ethyl | |
| 507 | 1 | m-nitro | H | 2-(1H- | H |
| | | phenyl | | imidazol-4- | |
| | | | | yl)-ethyl | |
| 508 | 1 | Cl | CH₂CH—O—R^{4b} | CH₂CH₂ | benzyl |
| 509 | 1 | Cl | CH(CH₃)CH₂—O—R^{4b} | CH(CH₃)CH₂ | benzyl |
| 510 | 1 | m-nitro | H | 2- | H |
| | | phenyl | | dimethylamino | |
| | | | | ethyl | |
| 511 | 2 | Cl | CH₂CH₂—O—R^{4b} | CH₂CH₂ | benzyl |
| 512 | 2 | Cl | H | 2-morpholin- | benzyl |
| | | | | 4-yl-ethyl | |

The compounds in Table VI were prepared suing the procedure outlined in Scheme I above.

Ex. 501

MS: found (MH+) 517.1, calc 516.21.

Ex. 502

MS :found (M−H) 493.0, calc 494.17

Ex. 503

MS: found (MH+) 525.2,calc 524.13.

Ex. 504

MS: found (MH+) 464.9, calc 464.16

Ex. 505

MS:found (MH+) 492.2, calc 491.18.

Ex. 506

MS:found (MH+) 461.6, calc 461.17.

Ex. 507

MS:found (MH+) 498.5, calc 497.18.

Ex. 508

MS:found (MH+) 477.1, calc 476.16.

Ex. 509

MS:found (MH+) 504.8, calc 504.19.

Ex. 510

MS:found (MH+) 475.1, calc 474.20

Ex. 511

¹H NMR (CD₃OD) δ: 2.43 (t, J=8 Hz, 2H,), 2.66 (t, J=8 Hz, 2H), 3.25 (m, 2H), 3.38 (m, 6H), 4.05 (s, 2H), 6.57 (d, J 10 Hz, 1H), 6.91 (d, J=2.5 Hz, 1H), 6.95-7.18 (m, 5H), 7.21 (d, J=2.5 Hz, 1H), 7.92 (d, J=10 Hz, 1H).

MS: found (MH+) 491.0, calc 490.18.

Ex. 512

¹H NMR (CD₃OD) δ: 2.44 (t, J=7 Hz, 2H), 2.76 (t, J=7 Hz, 2H), 3.0-3.24 (m, 4H), 3.48 (m, 4H), 3.75 (t, J=11 Hz, 2H), 3.99 (m, 2H), 4.13 (s, 2H), 6.66 (d, J=10 Hz, 1H), 7.00 (d, J=2 Hz, 1H), 7.02-7.26 (m, 5), 7.27 (d, J=2 Hz, 1H), 8.01 (d, J=10 Hz, 1H)

MS: found (MH+) 534.0, calc 533.22.

TABLE VII

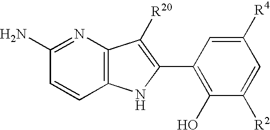

| Ex. | R² | R⁴ | R²⁰ |
|---|---|---|---|
| 601 | Cl | Ph | benzyl |
| 602 | Cl | Ph | H |
| 603 | Cl | o-cyano | H |
| | | phenyl | |
| 604 | Cl | o-carboxy | benzyl |
| | | phenyl | |
| 605 | Cl | o-carboxyphenyl | benzyl |
| 606 | Cl | p- | benzyl |
| | | methoxycarbonyl | |
| | | phenyl | |
| 607 | Cl | o,m-bis(methoxy | benzyl |
| | | carbonyl)phenyl | |

Compounds listed in Table VII were preapred by using the procedure outlined in Scheme VII.

Ex. 601

5-(5-Amino-3-benzyl-1H-pyrrolo[3,2-b]pyridin-2-yl)-3-chloro-biphenyl-4-ol

¹H-NMR (d₆-DMSO) δ ppm: 14.20 (bs, 1H), 12.27 (bs, 1H), 10.00 (bs, 1H), 8.06 (d, J=8.9 Hz, 1H), 7.78 (d, J=2.3 Hz, 1H), 7.66 (bs, 2H), 7.43-7.07 (m, 11H), 6.71 (d, J=9.1 Hz, 1H), 4.18 (s, 2H);

¹³C NMR (d₆-DMSO) δ ppm: 151.68, 150.07, 140.39, 137.80, 136.47, 132.33, 130.30, 129.39, 128.85, 128.34, 128.17, 127.83, 127.75, 127.34, 126.07, 125.86, 122.49, 122.39, 121.43, 106.56, 104.69, 28.49.

MS LCMS MH+ 426.14 (calc.), 426.6 (obs.).

Ex. 602:

5-(5-Amino-1H-pyrrolo[3,2-b]pyridin-2-yl)-3-chloro-biphenyl-4-ol

¹H-NMR (d₆-DMSO) δ ppm: 14.08 (bs, 1H), 12.47 (bs, 1H), 10.27 (bs, 1H), 8.07 (m, 2H), 7.80-7.70 (m, 4H), 7.47 (t, 3H), 7.38 (d, J=7.4 Hz, 1H), 7.10 (s, 1H), 6.69 (d, J=8.9 Hz, 1H);

¹³C NMR (d₆-DMSO) δ ppm: 151.34, 149.46, 138.19, 137.77, 133.16, 131.20, 129.26, 128.90, 127.54, 127.48, 126.52, 124.79, 123.08, 123.04, 120.94, 105.08, 96.39

MS LCMS (MH+) 336.09 (calc.), 336.2 (obs.).

Ex. 603:

5′-(5-Amino-1H-pyrrolo[3,2-b]pyridin-2-yl.)-3′-chloro-4′-hydroxy-biphenyl-2-carbonitrile

MS LCMS MH+ 361.09 (calc.), 360.9 (obs.).

Ex. 604:

5′-(5-Amino-1H-pyrrolo[3,2-b]pyridin-2-yl)-3′-chloro-4′-hydroxy-biphenyl-2-carboxylic acid (33).

MS LCMS MH+ 380.08 (calc.), 380.2 (obs.).

Ex. 605:

5′-(5-Amino-3-benzyl-1H-pyrrolo[3,2-b]pyridin-2-yl) -3′-chloro-4′-hydroxy-biphenyl-2-carboxylic acid

MS LCMS MH+ 470.13 (calc.), 470.2 (obs.).

Ex. 606:

5′-(5-Amino-3-benzyl-1H-pyrrolo [3,2-b]pyridin-2-yl) -3′-chloro-41-hydroxy-biphenyl-4-carboxylic acid methyl ester

MS: found (MH+) 484.4, calc. 484.13.

Ex. 607:

5′-(5-Amino-3-benzyl-1H-pyrrolo [3,2-b]pyridin-2-yl) -3′-chloro-41-hydroxy-biphenyl-2,5-dicarboxylic acid

MS: found (MH+) 514.4, calc. 514.11.

Utility

The compounds of this invention are useful as anticoagulants for the treatment or prevention of thromboembolic disorders in mammals. The term “thromboembolic disorders” as used herein includes arterial or venous cardiovascular or cerebrovascular thromboembolic disorders, including, for example unstable angina, first or recurrent ischemic attack, stroke, atherosclerosis, venous thrombosis, deep vein thrombosis, thrombophlebitis, arterial embolism, kidney embolisms, and pulmonary embolisms. The anticoagulant effect of compounds of the present invention is believed to be due to the inhibition of Factor Xa (FXa), Factor VIIa (FVIIa), and thrombin.

Factor Xa determinations were made in 50 mM Tris buffer, pH 7.5, containing 150 μM NaCl, 5 mM CaCl₂, 0.05% Tween-20, and 1.0 mM EDTA. Values of Ki app. were determined by allowing 2-4 nM human Factor Xa (Haematologic Technologies, VT, USA) to react with the 1 mM substrate (MeOC-Nle-Gly-Arg-pNA) in the presence of an inhibitor. Hydrolysis of the chromogenic substrate is followed spectrophotometrically at 405=m for five minutes. The enzyme assay routinely yielded linear progression curves under these conditions. Initial velocity measurements calculated from the progress curves by a kinetic analysis program (Batch Ki; Peter Kuzmic, BioKin, Ltd., Madison, Wis.) were used to determine Ki app. Compounds of the present invention are also useful as inhibitors of proteases, which play a significant role in the progression of cancer. Their inhibitory activity includes inhibition of urokinase (uPA) which has been postulated to have therapeutic value in treating cancer.

Some of the compounds of the present invention show selectivity between uPA and FXa, with respect to their inhibitory properties. The effectiveness of compounds of the present invention as inhibitors of Urokinase and Factor Xa is determined using synthetic substrates and purified Urokinase and purified human Factor Xa respectively.

The rates of hydrolysis by the chromogenic substrates were measured both in the absence and presence of compounds of the present invention. Hydrolysis of the substrates result in the release of the -pNA moiety, which is monitored spectrophotometrically by measuring the increase in absorbance at 405 nano meter (nm). A decrease in the rate of absorbance change at 405 nm in the presence of a inhibitor is indicative of enzyme inhibition. The results of this assay are expressed as the inhibitory constant, Ki app.

Definitions

The compounds of the present invention may have asymmetric centers. Compounds of the present invention containing an asymmetrically substituted atom may be isolated in optically active or racemic forms. It is well known in the art how to prepare optically active forms, such as by resolution of materials. Many geometric isomers of olefins, C═N double bonds, and the like can be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure (representing a compound of Formula I) are intended, unless the specific stereochemistry or isomeric form is specifically indicated.

As used herein, the following terms and abbreviations have the following meaning, unless indicated otherwise.

The term “prodrug” is intended to represent covalently bonded carriers which are capable of releasing the active ingredient of Formula I, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I include compounds wherein a hydroxy, amidino, guanidino, amino, carboxylic or a similar group is modified.

“Pharmaceutically acceptable salts” is as understood by one skilled in the art. Thus a pharmaceutically acceptable salt includes acid or base salts of compounds of Formula I. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is -understood that the pharmaceutically acceptable salts are non-toxic. Additional information on suitable pharmaceutically acceptable salts can be found in Remington 's Pharmaceutical Sciences, 17th ed., Mack Publishing Company, Easton, Pa., 1985, which is incorporated herein by reference.

“Optional” or “optionally” means that the subsequently described event or circumstance may or may not occur, and that the description includes instances where the event or circumstance occurs and instances in which it does not. For example, the phrase “optionally is substituted with one to three substituents” means that the group referred to may or may not be substituted in order to fall within the scope of the invention. Thus the term “optionally substituted” is intended to mean that any one or more hydrogens on a designated atom can be replaced with a selection from the indicated group, provided that the designated atom's normal valence is not exceeded, and that the substitution results in a stable compound. When the substituent is keto (═O) then 2 hydrogens on the atom are replaced. “Optional substituents” , unless otherwise indicated, are independently selected from a group consisting of H; N(R¹⁰)₂; NO₂; halogen; aryl; O—C₅₋₁₀ cyclo alkyl substituted with R¹⁰; guanidino; urea; thio urea; amidino; para or meta phenoxy; piperidin-4-yloxy; 4-amino-cyclohexyloxy; 1-(1-Imino-ethyl)-piperidin-4-yloxy; 1-(1-Imino-ethyl)-pyrrolidin-3-yloxy; 2-Amino-3-methyl-butyryl; 4-Acetimidoylamino-cyclohexyloxy; CO—C₁₋₄ alkyl, 1-(1-Imino-ethyl)-pyrrolidin-2-ylmethoxy; 2-(2-Hydroxycarbonimidoyl-pyridin-3-yloxy)-ethoxy; 3,4-Dicyano-phenoxy; SC₁₋₄ alkyl, S-aryl, pyrimidin-2-ol-5-yl, O—C₁₋₄ alkyl, COOR¹⁰, C(O)-pyrrolidine; C(O)CH(NH₂) CH₂OH; C(O)CH(NH₂) CH₂Ph; C(O)CH(NH₂)CH₂COOH; O-pyrrolidine; SO₂—C₁₋₄ alkyl, C(O)—(CH₂)₁₋₃-imidazole; SO₂—N(alkyl)₂; C(═N)—C₃; O-piperidine; 2-aminothiazol-5-ylmethoxy; O—CH₂—COOH; pyrrolidine-2-ylmethoxy; 2,4,6-triamino pyrimidin-5-ylmethoxy; NH—SO₂-alkyl; NHC₁-C₄ alkyl; N(C₁-C₄)₂ alkyl; CF₃; C₂₋₁₀ alkenyl and C₁₋₁₀ alkyl.

The term “alkyl”, as used herein, is intended to include branched and straight chain saturated aliphatic hydrocarbon groups having from 1 to 14 or the specified number of carbon atoms, illustrative examples of which include, but are not limited to, methyl, ethyl, n-propyl, i-propyl, n-butyl, i-butyl, sec-butyl, t-butyl, n-pentyl, and n-hexyl. “Alkenyl” is intended to include a branched or straight chain hydrocarbon group having one or more unsaturated carbon-carbon bonds which may occur in any stable point along the chain, such as ethenyl, propenyl, and the like. The term “alkelene” represents an alkyl group, as defined above, except that it has at least one center of unsaturation, i.e., a double bond. Illustrative examples are butene, propene, and pentene. The term “cycloalkyl”, “cycloalkyl ring”, “cycloalkyl radical” or “cyclic hydrocarbon” indicates a saturated or partially unsaturated three to fourteen carbon monocyclic or bicyclic hydrocarbon moiety which is optionally substituted with an alkyl group. Illustrative examples include cyclo propyl, cyclo hexyl, cyclo pentyl, and cyclo butyl. The term “alkoxy” as used herein represents —OC₁₋₆ alkyl.

The terms “Ar” and “aryl”, as used herein, are intended to represent a stable substituted or unsubstituted (collectively also referred to as ‘optionally substituted’) six to fourteen membered mono-, bi- or tri-cyclic hydrocarbon radical comprising carbon and hydrogen atoms. Illustrative examples are phenyl (Ph), naphthyl, anthracyl groups, and piperanyl. It is also intended that the terms “carbocycle” and “carbocyclic” include “Ar”, “aryl” as well as “cyclo alkyl” groups, which are defined above. “Halogen” or “halo”, as used herein, represents Cl, Br, F or I.

As used herein the term “bicyclic heterocyclic ring structure” is intended to represent a stable 7 to 10 membered bicyclic heterocyclic ring which is partially unsaturated or unsaturated (aromatic, i.e., heteroaryl) and which consists of carbon atoms and from 1 to 3 hetero atoms selected from S, O, and N, preferably nitrogen atoms. The nitrogen and sulfur atoms can exist in their respective oxidized states, while the nitrogen atom can also exist in its quaternized form. Illustrative examples of the bicyclic heterocyclic ring structure are 3H-imidazo[4,5-c]pyridine-2-yl, 1H-imidazo[4,5-c]pyridine-2-yl, 3H-pyrrolo[3,2-c]pyridine-2-yl, 3H-pyrrolo[3,2-c]pyrimidine-2-yl, thiazolo[5,4-c]pyridine-2-yl, oxazolo[5,4-c]pyridine-2-yl, 4H-thiopyrano[4,3-d]oxazole, 1H-indole-2-yl, 1H-benzimidazole-2-yl, 2,3-dihydro,1H-indole-2-yl, 2,5-dihydro-thiopyrano[2,3-b]pyrrole, thieno[2,3-c]pyridine, 4,5-dihydro-1H-benzoimidazole-2-yl, 1H-pyrrolo[2,3-c]pyridine, benzooxazole, 4H-thiopyrano[4,3-b]furan, 4,5-dihydrofuro[3,2-b]pyridine, 1,7-dihydro-thiopyrano-[2,3-b]pyrrole-2-yl, 1,4-dihydro-thiopyrano-[3,4-d]imidazole-2-yl, and 1,5-dihydro pyrano[2,3-d]imidazole-2-yl. It is preferred that when the total number of hetero atoms in the heterocycle exceeds 1, then the heteroatoms are not adjacent to one another. Preferred bicyclic heterocyclic ring structures comprise 9 to 10 membered bicyclic heterocyclic ring structures comprising a six membered ring and a five membered ring fused together such that the two rings have two common atoms. Illustrative examples of the preferred bicyclic heterocyclic ring structures are 1H-indole-2-yl, 1H-benzimidazole-2-yl.

The term “heteroaryl” is intended to represent a stable 5 to 10 membered aryl group (“aryl” as defined above), wherein one or more of the carbon atoms is replaced by a hetero atom selected from N, O, and S. The hetero atoms can exist in their chemically allowed oxidation states. Thus a Sulfur (S) atom can exist as a sulfide, sulfoxide, or sulfone. Preferred heteroaryl groups are six membered ring systems comprising not more than 2 hetero atoms. Illustrative examples of preferred heteroaryl groups are thienyl, N-substituted succinimide, 3-(alkyl amino)-5,5-dialkyl-2-cyclohexen-1-one, methylpyridyl, alkyl theophylline, tetrazolyl, furyl, pyrrolyl, indolyl, pyrimidinyl, isoxazolyl, purinyl, imidazolyl, pyridyl, pyrazolyl, quinolyl, and pyrazinyl. The term “heterocycloalkyl” means a stable cyclo alkyl group containing from 5 to 14 carbon atoms wherein one or more of the carbon atoms is replaced by a hetero atom chosen from N, O and S. The hetero atoms can exist in their chemically allowed oxidation states. Thus Sulfur (S) can exist as a sulfide, sulfoxide, or sulfone. The heterocycloalkyl group can be completely saturated or partially unsaturated. Illustrative examples are piperidine, 1,4-dioxane, and morpholine.

As used herein the terms “heterocyclyl”, “heterocyclic” and/or “het” are intended to represent a stable 5- to 7-membered monocyclic or 7- to 10-membered bicyclic heterocyclic ring which is saturated, partially unsaturated, or unsaturated (aromatic), which consists of carbon atoms and from one to 4 hetero atoms independently selected from a group consisting of N, O and S. The nitrogen and the sulfur hetero atoms can exist in their respective oxidized states. The heterocyclic ring may be attached to its pendent group at any heteroatom or carbon atom which results in a stable structure. The heterocyclic rings described herein may be substituted on a carbon or a nitrogen atom if the resulting compound is stable. The nitrogen in the heterocycle can exist in its quaternized form. It is preferred that when the total number of hetero atoms in the heterocycle exceeds 1, then the heteroatoms are not adjacent to one another. It is understood that the terms “heterocyclyl”, “heterocyclic”, and “het” include the terms “heteroaryl”, “heterocycloalkyl” and “bicyclic heterocyclic ring structure” as described above.

Preferred “heterocyclyl”, “heterocyclic” and/or “het” groups are selected from 1-(2,Hydroxymethyl-pyrrolidin-1-yl)-2,3-dimethyl-butan-1-one, 3-Pyridin-2-yl-propan-1-ol, N-(2,3-Dimethoxy-benzyl)-2-hydroxy-acetamide, 1-Methyl-2-m-tolyl-1H-benzoimidazole-5-carboxamidine, 2-Methyl-3,4,6,7-tetrahydro-imidazo[4,5-c]pyridine-5-carboxamidine, 2-Amino-3-hydroxy-1-(2-methyl-3,4,6,7-tetrahydro-imidazo[4,5-c]pyridin-5-yl)-propan-1-one, tetrazolyl, 2-Amino-1-(2-methyl-3,4,6,7-tetrahydro-imidazo[4,5-c]pyridin-5-yl)-ethanone, 2-Methyl-4,5,6,7-tetrahydro-3H-imidazo[4,5-c]pyridine, N-o-Tolyl-methanesulfonamide, 2-Methyl-benzothiazole, 3-Amino-1-(2-hydroxymethyl-pyrrolidin-1-yl)-propan-1-one, 2-Hydroxy-1-(2-hydroxymethyl-pyrrolidin-1-yl)-ethanone, 2-(2-Hydroxy-ethyl)-indan-1,3-dione, 5-Fluoro-2-methyl-1H-benzoimidazole, 2-Methyl-1H-imidazo[4,5-c]pyridine, 2-Hydroxy-N-(2-morpholin-4-yl-ethyl)-acetamide, 2-Methyl-1H-imidazo[4,5-b]pyridine, 2-Amino-1-(3-methyl-piperidin-1-yl)-ethanone, 2-Methyl-1H-benzoimidazol-4-ol, 2-Pyridin-2-yl-ethanol, N-(3-Hydroxy-propyl)-2-phenyl-acetamide, N-(3-Hydroxy-propyl)-3-phenyl-propionamide, N-(3-Hydroxy-propyl)-benzamide, N-(2-Hydroxy-ethyl)-2-phenyl-acetamide, (4-Hydroxy-butyl)-carbamic acid tert-butyl ester, (2-Hydroxy-ethyl)-carbamic acid benzyl ester, (4-Hydroxy-piperidin-1-yl)-phenyl-methanone, 4-Bromo-2-methoxy-benzylamine, 3-Methoxy-5-trifluoromethyl-benzylamine, N-(3,5-Dimethoxy-benzyl)-acetamide, 2-Methyl-1H-benzoimidazole-5-carboxamidine, and 2-Hydroxy-N-naphthalen-1-yl-acetamide.

The following structural representations further illustrate the term “het”:

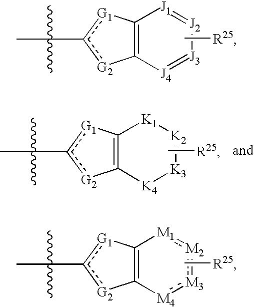

wherein G₁ and G₂ independently at each occurance represent S(O)₀₋₂, NH, N—R²⁴, O, CR¹⁰), or CHR¹⁰; J₁, J₂, J₃, and J₄ independently represent CR¹⁰ or N, wherein at least two of J₁, J₂, J₃, and J₄ represent CH; K₁, K₂, K₃ and K₄ independently represent —NHR¹⁰, —NHR²⁴, —CHR¹⁰, —CH—C(═NH)—NH₂, or N—C(═NH)—NH₂ wherein at least two of K₁, K₂, K₃ and K₄ represent CH₂; M₁, M₂, M₃ and M₄ independently represent—NHR¹⁰, —NHR²⁴, —CHR¹⁰, —CH—C (═NH)—NH₂, or N—C(═NH)—NH₂, wherein at least two of M₁, M₂, M₃ and M₄ represent CH or CH₂; and R²⁵ represents H, halogen, —C₁₋₆ alkyl, —NO₂, NHR¹⁰, NH—SO₂—R¹⁰, —OH, C₁₋₆ alkoxy, amidino, guanidino, —COOR¹⁰, or —CONHR¹⁰. The variables R¹⁰ and R²⁴ are as defined earlier. The dashed lines indicate optional unsaturation without violating the valency rules.

The term “basic group” as used under R⁷ and R⁸, defined earlier, is intended to represent amidino, guanidino, —C(═NH)N(R¹⁰)₂, 2-imidazoline, —N-amidinomorpholine, N-amidino piperidine, 4-hydroxy-N-amidino piperidine, N-amidino pyrrolidine, tetrahydro pyrimidine, and thiazolidin-3-yl-methylideneamine. The compounds of the present invention were named using the “Autonom”, a Beilstein Commander 2.1 Application, distributed by Beilstein.

The term “natural amino acid”, as used herein is intended to represent the twenty naturally occurring amino acids in their ‘L’ form, which are some times also referred as ‘common amino acids’, a list of which can be found in Biochemistry, Harper & Row Publishers, Inc. (1983). The term “unnatural amino acid”, as used herein, is intended to represent the ‘D’ form of the twenty naturally occurring amino acids described above. It is further understood that the term unnatural amino acid includes homologues of the natural amino acids, and synthetically modified form of the natural amino acids. The synthetically modified forms include amino acids having alkylene chains shortened or lengthened by up to two carbon atoms, amino acids comprising optionally substituted aryl groups, and amino acids comprised halogenated groups, preferably halogenated alkyl and aryl groups.

The term “natural amino acid side chain” is intended to represent a natural amino acid (“natural amino acid” as defined above) wherein a keto (C═O) group replaces the carboxylic acid group in the amino acid. Thus, for example, an alanine side chain is C(═O)—CH(NH₂)—CH₃; a valine side chain is C(═O)—CH(NH₂)—CH(CH₃)₂; and a cysteine side chain is C(═O)—CH(NH₂)—CH₂—SH. The term “unnatural amino acid side chain” is intended to represent an unnatural amino acid (“unnatural amino acid” as defined above) wherein a keto (C═O) group replaces the carboxylic acid group forming unnatural amino acid side chains similar to ones illustrated under the definition of “natural amino acid side chain” above.

It thus follows that a “N-natural amino acid side chain” substituent and “N-unnatural amino acid side chain” substituent, which can represent Q, Q¹, Q², Q³, L¹, L², L³ and L⁴, is a group wherein the nitrogen atom (N) is the annular ring atom substituted with a natural or unnatural amino acid side chain (natural or unnatural amino acid side chain is a defined above). The point of attachment between the nitrogen atom and the natural or unnatural amino acid side chain is at the keto (C═O) group of the respective amino acids. Thus a N-natural amino acid, i.e., N-cysteine, is N—C(═O)—CH(NH₂)—CH₂—SH.

1. A compound of Formula I: 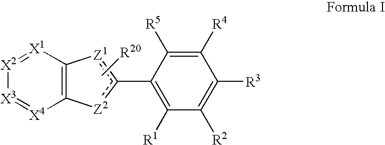
its prodrug forms, or pharmaceutically acceptable salts thereof, wherein
R¹ represents OH, halogen, COOH, COO—C₁₋₄ alkyl, O—(CH₂)₀₋₁—Ar, N(R¹⁰),₂ CH₂OR¹⁰, C₁₋₆ halogenated alkyl, O—(CH₂)₁₋₄—CO—N(R¹⁰)₂, SC₁₋₄alkyl, NHSO₂C₁₋₄alkyl, SO₂—OH, O—SO₂—OH, O—C₁₋₄ alkyl, O—C₃₋₉ cyclo alkyl, O—SO₂—O—C₁₋₄ alkyl, OP(O)(OH)₂, or OPO₃C₁₋₄ alkyl; R², R³, R⁴, and R⁵ independently at each occurance represent H, SH, OR¹⁰, halogen, COOR¹⁰, (CH₂)₀₋₆—CONR¹¹R¹², optionally substituted aryl, optionally substituted heterocyclyl, C₄₋₁₄ cycloalkyl-C₁₋₄ alkyl, C₁₋₄ alkyl aryl, optionally substituted C₁₋₁₄ straight chain, branched or cyclo alkyl, O—(CH₂)₂₋₆—NR¹⁰—(CH₂)₀₋₃—R²⁴, NR¹⁰R²⁴, (CH₂)₁₋₆—NR³³R³⁴ (CH₂)₁₋₆—COOR³³, O—(CH₂)₁₋₃—CO—-het, O—(CH₂)₁₋₂—NH—CO-aryl, O—(CH₂)₁₋₂—NR¹⁰-CO—NR¹⁰R³³, (CH₂)₁₋₄—CONR¹⁰(CH₂)₁₋₄-heterocyclyl, O—(CH₂)₀₋₂—C(O)—NR³³R³⁴, O—(CH₂)₁₋₄—COOR¹⁰, O—(CH₂)₁₋₃-het-R³², O-optionally substituted cycloalkyl, O—(CH₂)₁₋₄—NR¹⁰—COO-t-butyl, O—(CH₂)₁₋₄—NR¹⁰R³³, O—(CH₂)₁₋₄—NR¹⁰—C(O)—C₀₋₃-alkyl-optionally substituted aryl, O-substituted cycloalkyl, O—(CH₂)₀₋₆-optionally substituted aryl, (CH₂)₁₋₄—NH—C(O)O—(CH₂)₁₋₄-PhR¹³R¹⁴, NO₂, O—(CH₂)₀₋₄—C(O)—NH-tetrahydro carboline, NR¹⁰R²⁸, O—(CH₂)₁₋₃-optionally substituted het, CH₂COOCH₃, CH═CH—COOCH₃, 5-amidino benzimidazole, SO₂—N(R¹⁰)₂, 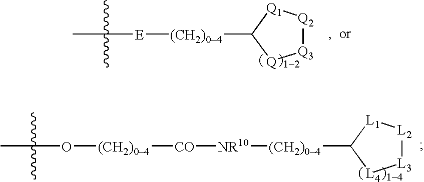
alternatively R² and R³, R³ and R⁴ or R⁴ and R⁵ can be taken together to form 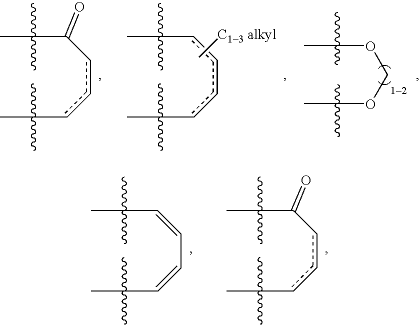
X¹ represents C—R⁶, N or N—O;
X² represents C—R⁷, N or N—O;
X³ represents C—R⁸, N or N—O;
X⁴ represents C—R⁹, N or N—O;
Z¹ and Z² independently at each occurance represent C or N;
R⁶, R⁸ and R⁹ independently at each occurance represents H, halogen, cyano, C₁₋₄ alkyl, C₁₋₄ halogenated alkyl, NO₂, O-aryl or OR¹¹, CF₃, OC₁₋₄ alkyl, (CH₂)₀₋₄-aryl, (CH₂)₀₋₄-heteroaryl, or (CH₂)₀₋₄-heterocyclyl;
R⁷ represents NH₂, NHR¹⁰, N(R¹⁰) ₂, NHSO₂—C₁₋₁₄ alkyl, NHSO-aryl, OH, NHCO—C₁₋₁₄ alkyl, NHNH₂, NHOH, NHCO—C₁₋₁₄ alkyl, NR¹⁰NH₂, NHN(R¹⁰)₂, NH(C═NH)NH₂, NH(C═O)N(R¹⁰)₂; alternatively R⁶ and R⁷, R⁷ and R⁸, R⁸ and R⁹, along with the respective carbon atoms to which they are attached, can be taken together to represent a 5 to 10 atom saturated, partially saturated or aromatic, carbocyclic or heterocyclic ring structure substituted with R⁴¹;
R¹⁰ independently at each occurance represents H, (CH₂)₀₋₂-aryl, C₁₋₄ halo alkyl, or C₁₋₁₄ straight chain, branched or cyclo alkyl, and alternatively, when one atom is substituted with two R¹⁰ groups, the atom along with the R¹⁰ groups can form a five to 10 cycloalkyl, heterocyclyl or aryl group;
R¹¹ and R¹² independently at each occurance represent H or C₁₋₄ alkyl, (CH₂)₁₋₄—OH, (CH₂)₁₋₄—OC₁₋₄alkyl, (CH₂)₀₋₄-aryl, (CH₂)₁₋₄—(R¹⁰)₂;
R²⁰ represents R²⁴, C₁₋₄-alkyl, (CH₂)₁₋₃-biphenyl, (CH₂)₁₋₄-Ph-N(SO₂—C₁₋₂-alkyl)₂, (CH₂)₁₋₄—NH—C(O)R²⁴, (CH₂)₁₋₄—NH—SO₂—R²⁴, halogen, COOR¹⁰, (CH₂)₁₋₄-Ph-N(SO₂—C₁₋₂alkyl), (CH₂)₁₋₄—NR¹⁰—C(O)—R²⁴, (CH₂)₁₋₄—NR¹⁰—SO₂—R²⁴, (CH₂)₁₋₄-het, (CH₂)₁₋₄—CON(R¹⁰)₂, (CH₂)₁₋₄—N(R¹⁰)—C(O)—NR¹⁰R²⁴, (CH₂)₁₋₄—N(R¹⁰)—C(S)—NR¹⁰R²⁴ or (CH₂)₁₋₃—COOH;
R²⁴ represents R¹⁰, (CH₂)₁₋₄-optionally substituted aryl, (CH₂)₀₋₄OR¹⁰, CO—(CH₂)₁₋₂—N(R¹⁰)₂, CO(CH₂)₁₋₄—OR¹⁰, (CH₂)₁₋₄—COOR¹⁰, (CH₂)₀₋₄—N(R¹⁰)₂, SO₂R¹⁰, COR¹⁰, CON(R¹⁰)₂, (CH₂)₀₋₄-aryl-COOR¹⁰, (CH₂)₀₋₄-aryl-N(R¹⁰)₂, or (CH₂)₁₋₄-het-aryl;
R²⁸ represents (CH₂)₁₋₂-Ph-O—(CH₂)₀₋₂-het-R³⁰, C(O)-het, CH₂-Ph-CH₂-het-(R³⁰)₁₋₃; (CH₂)₁₋₄-cyclohexyl-R³¹, CH₂-Ph-O-Ph-(R³⁰)₁₋₂, CH₂—(CH₂OH) -het-R³⁰, CH₂-Ph-O-cycloalkyl-R³¹, CH₂-het-C(O)—CH₂-het-R³⁰, or CH₂-Ph-O—(CH₂)—O-het-R³⁰;
R³⁰ represents SO₂N(R¹⁰)₂, H, NHOH, amidino, or C(═NH)CH₃;
R³¹ represents R³⁰, amino-amidino, NH—C(═NH)CH₃ or R¹⁰;
R³² represents H, C(O)—CH₂—NH₂, or C(O)—CH(CH(CH₃)₂)—NH₂;
R³³ and R³⁴ independently at each occurance represent R¹⁰, (CH₂)₀₋₄—Ar, optionally substituted aryl, (CH₂)₀₋₄ optionally substituted heteroaryl, (CH₂)₁₋₄—CN, (CH₂)₁₋₄—N(R¹⁰)₂, (CH₂)₁₋₄—OH, (CH₂)₁₋₄—SO₂—N(R¹⁰)₂;
alternatively, R³³ and R³⁴ along with the nitrogen atom that they are attached to forms a 4 to 14 atom ring structure selected from tetrahydro-1H-carboline; 6,7-Dialkoxyoxy-2-substituted 1,2,3,4-tetrahydro-isoquinoline, 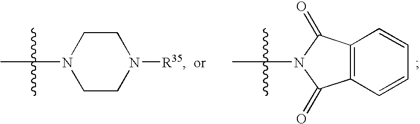
R³⁵ represents R¹⁰, SO₂—R¹⁰, COR¹⁰, or CONHR¹⁰;
E represents a bond, S(O)₀₋₂, O or NR¹⁰;
Q, Q¹, Q², Q³, L¹, L², L³ and L⁴ independently at each occurance represent N-natural or unnatural amino acid side chain, CHR¹⁰, O, NH, S(O)₀₋₂, N—C(O)—NHR¹⁰, SO₂—N(R¹⁰)₂, N—C(O)—NH—(CH₂)₁₋₄—R²⁶, NR¹⁰, N-heteroaryl, N—C(═NH)—NHR¹⁰, or N—C(═NH)C₁₋₄ alkyl;
R²⁶ represents OH, NH₂, or SH;
R⁴¹ represents NH₂, NHR¹⁰, or N(R¹⁰)₂, NHNH₂, NHOH, NR¹⁰NH₂, NHN(R¹⁰)₂, NH(C═NH)NH₂,NH(C═O)N(R¹⁰)₂;
provided that, (i) not all of X¹, X², X³ and X⁴ represent N or N—O simultaneously.

2. A compound of claim 1, wherein
R¹ represents OH, halogen or COOH;
R², R³, R⁴ and R⁵ independently at each occurance represent H, SH, OR¹⁰, halogen, COOR¹⁰, (CH₂)₀₋₄—CONR¹¹R¹², optionally substituted aryl, optionally substituted heterocyclyl, C₄₋₁₄ cycloalkyl-C₁₋₄ alkyl, C₁₋₄ alkyl aryl, optionally substituted C₁₋₁₄ straight chain, branched or cyclo alkyl, O—(CH₂)₂₋₆—NR¹⁰—(CH₂)₀₋₃—R²⁴, NR¹⁰R²⁴, (CH₂)₁₋₄—NR³³R³⁴, (CH₂)₁₋₄—COOR³³, O—(CH₂)₁₋₃—CO—het, O—(CH₂)₁₋₂-NH—CO-aryl, O—(CH₂)₁₋₂—NR¹⁰—CO—NR¹⁰R³³, O—(CH₂)₀₋₂—C(O)—NR³³R³⁴, O—(CH₂)₁₋₄—COOR¹⁰, O—(CH₂)₁₋₃-het-R³², O-optionally substituted cycloalkyl, O—(CH₂)₁₋₄—NR¹⁰—COO-t-butyl, O—(CH₂)₁₋₄—NR¹⁰R³³, O—(CH₂)₁₋₄—NR¹⁰—C(O)—C₀₋₃-alkyl-optionally substituted aryl, O-substituted cycloalkyl, O—(CH₂)₀₋₆-optionally substituted aryl, (CH₂)₁₋₄—NH—C(O)O—(CH₂)₁₋₄-PhR¹³R¹⁴, NO₂, O—(CH₂)₀₋₄—C(O)—NH-tetrahydro carboline, NR¹⁰R²⁸, O—(CH₂)₁₋₃—optionally substituted het, CH₂COOCH₃, CH═CH—COOCH₃, 5-amidino benzimidazole, 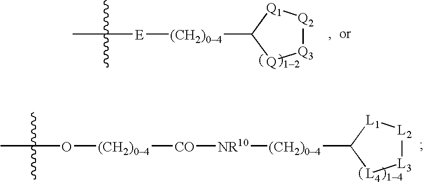
alternatively R² and R³ taken together form 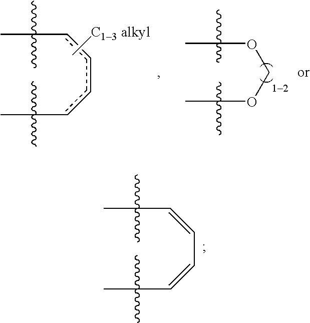
X¹ represents C—R⁶, N or N—O;
X² represents C—R⁷;
X³ represents C—R⁸;
X⁴ represents C—R⁹;
Z¹ represents C;
Z² represents N;
R⁶, R⁸ and R⁹ independently at each occurance represents H, halogen, cyano, C₁₋₄ alkyl, C₁₋₄ halogenated alkyl, NO₂, O-aryl or OR¹¹;
R⁷ represents NH₂, NHR¹⁰, N(R¹⁰)₂, NHSO₂—C₁₋₁₄ alkyl, NHSO-aryl, OH, NHCO—C₁₋₁₄ alkyl, NHNH₂, NHOH, NHCO—C₁₋₁₄ alkyl, NR¹⁰NH₂, NHN(R¹⁰)₂, NH(C═NH)NH₂, NH(C═O)N(R¹⁰)₂; alternatively
R⁶ and R⁷, R⁷ and R⁸, R⁸ and R⁹, along with the respective carbon atoms to which they are attached, can be taken together to represent a 6 saturated or aromatic, carbocyclic or heterocyclic ring structure substituted with R⁴¹;
R²⁰ represents R²⁴, C₁₋₄-alkyl, (CH₂)₁₋₃-biphenyl, (CH₂)₁₋₄-Ph-N(SO₂-C₁₋₂-alkyl)₂, (CH₂)₁₋₄—NH—C(O)—R²⁴, (CH₂)₁₋₄—NH—SO—R²⁴, halogen, COOR¹⁰, (CH₂)₁₋₄-Ph-N(SO₂—C₁₋₂alkyl), (CH₂)₁₋₄—NR¹⁰—C(O)—R²⁴, (CH₂)₁₋₄—NR¹⁰—SO₂—R²⁴, (CH₂)₁₋₄-het, (CH₂)₁₋₄—CON(R¹⁰)₂, (CH₂)₁₋₄—N(R¹⁰)—C(O)—NR¹⁰R²⁴, (CH₂)₁₋₄—N(R¹⁰)—C(S)—NR¹⁰R²⁴, or (CH₂)₁₋₃—COOH;
R²⁴ represents R²⁴, (CH₂)₁₋₄-optionally substituted aryl, (CH₂)₀₋₄—OR¹⁰, CO— (CH₂)₁₋₂—N(R¹⁰)₂, CO(CH₂)₁₋₄—OR¹⁰, (CH₂)₁₋₄—COOR¹⁰, (CH₂)₀₋₄—N(R¹⁰)₂, SO₂R¹⁰, COR¹⁰, CON(R¹⁰)₂, (CH₂)₀₋₄-aryl-COOR¹⁰, (CH₂)₀₋₄-aryl-N(R¹⁰)₂, or (CH₂)₁₋₄-het-aryl;
R²⁸ represents (CH₂)₁₋₂-Ph-O— (CH₂)₀₋₂-het-R³⁰, C(O)-het, CH₂-Ph-CH₂-het-(R³⁰)₁₋₃; (CH₂)₁₋₄-cyclohexyl-R³¹, CH₂-Ph-O-Ph-(R³⁰)₁₋₂, CH₂—(CH₂OH)-het-R³⁰, CH₂-Ph-O-cycloalkyl-R³¹, CH₂-het-C(O)—CH₂-het-R³⁰, or CH₂-Ph-O—(CH₂) —O-het-R³⁰;
R³⁰ represents SO₂N(R¹⁰)₂, H, NHOH, amidino, or C(═NH)CH₃;
R³¹ represents R³⁰, amino-amidino, NH—C(═NH)CH₃ or R¹⁰;
R³² represents H, C(O)—CH₂—NH₂, or C(O)—CH(CH(CH₃)₂)—NH₂;
R³³ and R³⁴ independently at each occurance represent R¹⁰, (CH₂)₀₋₄—Ar, optionally substituted aryl, (CH₂)₀₋₄ optionally substituted heteroaryl, (CH₂)₁₋₄—CN, (CH₂)₁₋₄—N(R¹⁰)₂, (CH₂)₁₋₄—OH, (CH₂)₁₋₄—SO₂—N(R¹⁰)₂;
alternatively, R³³ and R³⁴ along with the nitrogen atom that they are attached to forms a 4 to 14 atom ring structure selected from tetrahydro-1H-carboline; 6,7-Dialkoxyoxy-2-substituted 1,2,3,4-tetrahydro-isoquinoline, 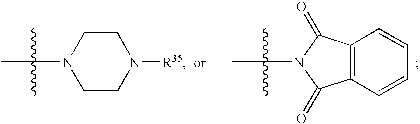
R³⁵ represents R¹⁰, SO₂—R¹⁰, COR¹⁰, or CONHR¹⁰;
E represents a bond, S(O)₀₋₂, O or NR¹⁰;
W₁, W₂, W₃ and W, independently represent C or N; and
Q, Q¹, Q² Q³, L¹, L², L³ and L⁴ independently at each occurance represent N-natural or unnatural amino acid side chain, CHR¹⁰, O, NH, S(O)₀₋₂, N—C(O)—NHR¹⁰, SO₂—N(R¹⁰)₂, N—C(O)—NH—(CH₂)₁₋₄—R²⁶, NR¹⁰, N-heteroaryl, N—C(═NH)—NHR¹⁰, or N—C(═NH)C₁₋₄ alkyl;
R²⁶ represents OH, NH₂, or SH; and
provided that, (i) not all of X¹, X², X³ and X⁴ represent N or N—O simultaneously.

3. A compound of claim 2 wherein
R¹ represents OH, O-Ph, COOH, or P(O)(OH)₂;
R² represents H, halo, optionally substituted alkyl or optionally substituted aryl or heteroaryl;
R³ represents C₀₋₆ alkyl-COOH;
R⁵ represents H, C₁₋₄ alkyl or OR¹⁰;
X¹ represents N or N—O;
R⁷ represents NH₂ or NHC₁₋₃ alkyl;
R²⁰ represents H, C₁₋₂ alkyl, (CH₂)₁₋₄-optionally substituted aryl, (CH₂)₁₋₄-het; (CH₂)₁₋₄—N(R¹⁰)₂, (CH₂)₁₋₄—CON(R¹⁰)₂, (CH₂)₁₋₄—NR¹⁰—C(O)—R²⁴, (CH₂)₁₋₄—NR¹⁰—SO₂—R²⁴, or (CH₂)₁₋₃—COOH.

4. A compound of claim 3 wherein
R⁴ represents (CH₂)₀₋₆—COOR¹⁰, optionally substituted heteroaryl, (CH₂)₀₋₄—CONR¹⁰R¹¹, C₁₋₁₀-straight chain alkyl, branched alkyl or cycloalkyl group substituted with 1-3 groups selected from COOR¹⁰, CONHR¹⁰, OR¹⁰, or aryl.

5. A compound of claim 4 wherein
R¹ represents OH or COOH.

6. A compound of claim 2 wherein
R¹ represents OH;
R² represents Cl, Br, 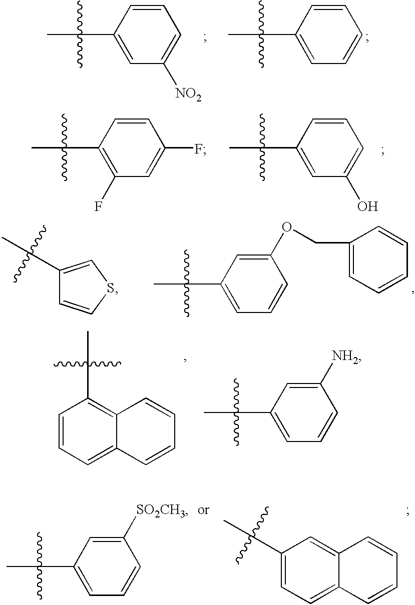
R³ represents H;
R⁴ represents CH₂COOH, CH₂CONH₂, CH₂COO—C₂H₅, CH₂CH₂COOH, Br, COOH, CH₂CON(CH₂CH₂OH)₂, CH₂CONHCH₂CH₂OCH₃, CH₂CONHCH₂CH₂OCH₃, methyl, COOCH₃, CH(CH₃)COOH, 1-tetrazolyl, SO₂NH₂₁ 2-carboxy-phenyl-1-y, phenyl, SO₂OH, 2-methyl-phenyl-1-yl, 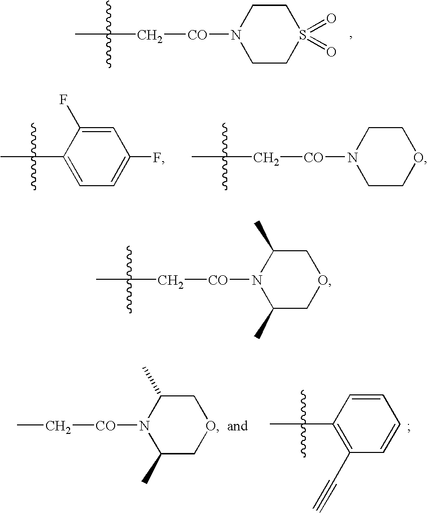
R⁵ represents H;
X¹ represents N;
X² represents CR⁷;
X² represents CR⁸;
X⁴ represents CR⁹;
R⁷ represents NH₂;
R⁸ represents methyl, chloro, H. OH or methoxy;
R⁹ represents H or methyl;
Z¹ represents C;
Z² represents N; and
R²⁰ represents benzyl, H, CH(Br)Ph, CH₂-Ph-p-Cl, or CH₂-pyridino.

7. A compound of claim 6 wherein
R¹ represents OH;
R² represents H, halogen, OH, phenyl, heteroaryl or substituted phenyl; and
R⁴ represents H, halo, (CH₂)₀₋₄—COOR¹⁰, (CH₂)₀₋₄—CONH₂, (CH₂)₀₋₄—CONHR³³, (CH₂)₀₋₄-heteroaryl, C₁₋₈ branched alkylene-COOR¹⁰, or C₂₋₆ alkenelyne-COOR¹⁰; and
R²⁰ represents H or (CH₂)₀₋₃-optionally substituted phenyl, (CH₂)₀₋₃-ar-yl or (CH₂)₀₋₃-heteroaryl.

8. A compound of claim 2, wherein the compound is selected from
[3-(5-Amino-3-benzyl-1H-pyrrolo[3,2-b]pyridin-2-yl)-5-chloro-4-hydroxy-phenyl]-acetic acid ethyl ester;
8-(5-Amino-3-benzyl-1H-pyrrolo[3,2-b]pyridin-2-yl)-6-bromo-7-hydroxy-3,4-dihydro-2H-naphthalen-1-one;
3-[3-(5-Amino-3-benzyl-1H-pyrrolo[3,2-b]pyridin-2-yl)-5-chloro-4-hydroxy-phenyl]-propionic acid;
[5-(5-Amino-3-benzyl-1H-pyrrolo[3,2-b]pyridin-2-yl)-6-hydroxy-31-nitro-biphenyl-3-yl]-acetic acid;
[3-(5-Amino-3-benzyl-1H-pyrrolo[3,2-b]pyridin-2-yl)-5-chloro-4-hydroxy-phenyl]-acetic acid;
2-[3-(5-Amino-3-benzyl-1H-pyrrolo[3,2-b]pyridin-2-yl)-5-chloro-4-hydroxy-phenyl]-acetamide;
2-[3-(5-Amino-3-benzyl-1H-pyrrolo[3,2-b]pyridin-2-yl)-5-chloro-4-hydroxy-phenyl]-N-(2-morpholin-4-yl-ethyl)-acetamide;
2-(5-Amino-1H-pyrrolo[2,3-c]pyridin-2-yl)-4,6-dichloro-phenol;
8-(5-Amino-3-benzyl-1H-pyrrolo[3,2-b]pyridin-2-yl)-6-bromo-7-hydroxy-3,4-dihydro-2H-naphthalen -1-one;
3-[3-(5-Amino-3-benzyl-1H-pyrrolo[3,2-b]pyridin-2-yl)-5-chloro-4-hydroxy-phenyl]-propionic acid;
[5-(5-Amino-3-benzyl-1H-pyrrolo[3,2-b]pyridin-2-yl)-6-hydroxy-3′-nitro-biphenyl-3-yl]-acetic acid;
[3-(5-Amino-1H-pyrrolo[3,2-b]pyridin-2-yl)-5-chloro-4-hydroxy-phenyl]-acetic acid;
[5-(5-Amino-1H-pyrrolo[3,2-b]pyridin-2-yl)-6-hydroxy-31-nitro-biphenyl-3-yl]-acetic acid;
2-[5-(5-Amino-1H-pyrrolo [3,2-b]pyridin-2-yl)-6-hydroxy-3′-nitro-biphenyl-3-yl]-N-(2-morpholin-4-yl-ethyl)-acetamide;
2-[5-(5-Amino-1H-pyrrolo[3,2-b]pyridin-2-yl)-6-hydroxy-3′-nitro-biphenyl-3-yl]-N-(2-methoxy-et hyl)-acetamide; and
2-[5-(5-Amino-1H-pyrrolo [3,2-b]pyridin-2-yl)-6-hydroxy-3′-nitro-biphenyl-3-yl]-N-(2-dimethylamino-ethyl)-acetamide.

9. A pharmaceutical composition comprising a pharmaceutically acceptable carrier and a therapeutically effective amount of a compound according to claim 1 or a pharmaceutically acceptable salt thereof.

10. A pharmaceutical composition comprising a pharmaceutically acceptable carrier and a therapeutically effective amount of a compound according to claim 2 or a pharmaceutically acceptable salt thereof.

11. A method for treating or preventing a thromboembolic disorder, comprising administering to a patient in need thereof a therapeutically effective amount of a compound according to claim 2 or a pharmaceutically acceptable salt thereof.

12. A compound of claim 2 wherein:
X¹ represents C—R⁶;
X² represents C—R;
X³ represents N or N—O;
X⁴ represents C—R⁹;
Z¹ represents C; and
Z² represents N.

13. A compound of claim 12 wherein
R¹ represents OH, COOH, or P(O)(OH)₂;
R² represents H, halo, optionally substituted alkyl or optionally substituted aryl or heteroaryl;
R³ represents C₀₋₆ alkyl-COOH;
R⁵ represents H, C₁₋₄ alkyl or OR¹⁰;
X¹ represents N or N—O;
R⁷ represents NH₂ or NHC₁₋₃ alkyl;
R²⁰ represents H, C₁₋₂ alkyl, (CH₂)₁₋₄-optionally substituted aryl, (CH₂)₁₋₄-het; (CH₂)₁₋₄—N(R¹⁰)₂, (CH₂)₁₋₄—CON(R¹⁰)₂, (CH₂)₁₋₄—NR¹⁰—C(O)—R²⁴, (CH₂))₁₋₄—NR¹⁰—SO₂—R²⁴, or (CH₂)₁₋₃—COOH.

14. A compound of claim 13 wherein
R⁴ represents (CH₂)₀₋₆—COOR¹⁰, optionally substituted heteroaryl, (CH₂)₀₋₄—CONR¹⁰R¹¹, C₁₋₁₀-straight chain alkyl, branched alkyl or cycloalkyl group substituted with 1-3 groups selected from COOR¹⁰, CONHR¹⁰, or OR¹⁰.

15. A compound of claim 14 wherein
R¹ represents OH or COOH.

16. A compound of claim 22 wherein
R⁷ represents NH_{2.}

17. A compound of claim 16 wherein
R¹ represents OH;
R² represents H, halogen, OH, phenyl heteroaryl, or substituted phenyl; and
R⁴ represents H, halo, (CH₂)₀₋₄—COOR¹⁰, (CH₂)₀₋₄—CONH₂, (CH₂)₀₋₄—CONHR³³, (CH₂)₀₋₄-heteroaryl, C₁₋₈ branched alkylene-COOR¹⁰, or C₂₋₆ alkenyl-COOR¹⁰; and
R²⁰ represents H or (CH₂)1-3-optionally substituted phenyl.

18. A compound of claim 12, wherein the compound is selected from
2-(5-Amino-1H-pyrrolo[2,3-c]pyridin-2-yl)-4,6-dichloro-phenol; and
2-(5-Amino-3-benzyl-1H-pyrrolo[2,3-c]pyridin-2-yl)-4,6-dichloro-phenol.

19. A pharmaceutical composition comprising a pharmaceutically acceptable carrier and a therapeutically effective amount of a compound according to claim 12 or a pharmaceutically acceptable salt thereof.

20. A pharmaceutical composition comprising a pharmaceutically acceptable carrier and a therapeutically effective amount of a compound according to claim 12 or a pharmaceutically acceptable salt thereof.

21. A method for treating or preventing a thromboembolic disorder, comprising administering t a patient in need thereof a therapeutically effective amount of a compound according to claim 12 or a pharmaceutically acceptable salt thereof.
